(19) United States
(12) Patent Application Publication
(10) Pub. No.: US 2022/0380331 A1
(43) Pub. Date: Dec. 1, 2022

(54) TREATMENT OF CONDITIONS OF THE NERVOUS SYSTEM

(71) Applicants: Jessica KWOK, Bradford, Leeds (GB); Richard FOSTER, Bradford, Leeds (GB); James DUNCAN, Bradford, Leeds (GB)

(72) Inventors: Jessica KWOK, Bradford, Leeds (GB); Richard FOSTER, Bradford, Leeds (GB); James DUNCAN, Bradford, Leeds (GB)

(21) Appl. No.: 17/771,758

(22) PCT Filed: Oct. 25, 2020
(86) PCT No.: PCT/EP2020/079979

§ 371 Date: Apr. 25, 2022

Related U.S. Application Data
(60) Provisional application No. 62/926,001, filed on Oct. 25, 2019.

(51) Int. Cl. C07D 311/16; A61P 25/28

(52) U.S. Cl. CPC C07D 311/16; A61P 25/28

(57) ABSTRACT

Provided is 4-methylumbelliferone, derivatives and salts thereof, or a composition comprising same for use in the treatment of a condition of the nervous system in a subject. Preferably, the condition is one associated with a scar, such as a glial scar. Typically, the condition of the nervous system is selected from the group comprising conditions caused by trauma, injury, infection, degeneration, structural defects, tumours, blood flow disruption. The neural and other lesions are made permeable and repairable.

### FIELD OF THE INVENTION

The current invention relates to treatment of conditions of the nervous system in a subject. In particular, the current invention relates to treatment of a spinal cord injury in a subject. The invention relates to treatment of lesions associated with conditions of the nervous system.

### BACKGROUND OF THE INVENTION

The spinal cord is a long tubular structure comprising nervous tissue and it functions primarily to transmit nerve signals, or messages, allowing the body and the brain to communicate. It is also responsible for coordinating reflexes. The spinal cord is encased within the spinal vertebral column. The spinal nerves are located in spaces between the vertebrae and the nerve roots exit the spinal cord either side of each vertebrae. The spinal cord is divided into segments and major body functions correspond to specific segments. There are 31 spinal cord nerve segments in the human spinal cord.

Spinal cord injury (SCI) is damage to the spinal cord that causes changes in its function. These changes may be temporary or permanent. Injury can occur at any level of the spinal cord and can be complete or incomplete. Functional loss, both motor and sensory, depends on the site of injury and the extent of the damage.

Dislocation, or fracturing, of a vertebra by traumatic injury can cause the spinal cord to be contused or transected. This primary mechanical injury causes initial damage to the spinal cord, disrupting the blood supply and causing damage to cells and neurones. The secondary response follows and causes destruction of the central matter of the spinal cord. Loss of myelin results in loss of signal transduction. Glial cells migrate to the site of the spinal cord injury in an attempt to repair the damage caused. A glial scar then forms around the injury site and prevents regeneration. Glial scar is composed of two components, cellular and biochemical. Astrogliosis and microglia activation happens after injury to the spinal cord. They secrete a plethora of molecules, including chondroitin sulphate proteoglycans.

There are two million cases or more of spinal cord contusion injury. There are six million cases or more of Stroke. There are seven hundred thousand cases or more of cerebral palsy. There are one million cases or more of Parkinson's disease. There are four hundred thousand cases or more of multiple sclerosis.

Current treatment methods for spinal cord injury intravenously include injecting a steroid called methylprednisolone within 8 hours of injury, but this has proven to be of limited or no curative benefit.

Neuroplasticity is a process which the central nervous system (CNS) adapts to changes from the external environment through re-organisation of synaptic connections and circuitries. It is crucial for the successful functional recovery after a spinal cord injury. Perineuronal nets (PNNs) are dense pericellular extracellular matrix structures found throughout the CNS (Kwok et al., 2011) and their formation has been linked with the termination of developmental plasticity (Carulli et al., 2010; Pizzorusso et al., 2002). PNNs wrap the surface of neurones, with holes where synapses locate (FIG. 7). This means that it is less likely for these neurones to have synapses for functional recovery. This limits synapse formation and thus neuroplasticity.

Chondroitin sulphate proteoglycans (CSPGs) are a family of glycans which inhibit neurite outgrowth and thus regeneration and plasticity (Kwok et al., 2011; Silver and Miller, 2004). CSPGs have been found to be present in PNNs and up-regulated in glial scars after spinal cord injury.

Removal of CSPGs in the PNNs and glial scar via injection of chondroitinase ABC (ChABC) has been demonstrated to open a window of plasticity and regeneration to promote recovery after SCI, in both acute and chronic injury models of up to 18 months (Bradbury et al., 2002; Wang et al., 2011; Warren et al., 2018). ChABC has proven beneficial to recovery alongside other treatments, including rehabilitation (Garcia-Alias et al., 2011). However, this approach poses significant hurdles in its translation to human application (e.g. limited stability as an enzyme thus needs repeated injection, the injection is invasive, potential development of immune response due to prolonged exposure to a bacterial protein).

4-methylumbelliferone is a compound commonly used in bile therapy. It is available under the name Hymecromone in Europe. The drug has been used for many years in this area and has an excellent safety profile. The compound was first used in vitro in 1995 by Nakamura et al., to inhibit HA-synthesis in skin fibroblasts (Nakamura et al., 1995).

Fontaine et al., conducted a toxicological and teratological study of 4-methylumbelliferone (Fontaine et al., 1968). They reported results of studies in acute toxicity, chronic toxicity, local tolerance and experimental teratogenesis in several species. In studies of chronic toxicity, the maximum tolerated dose on the oral rate was found to be equal to 6000 mg/kg in rats. In the chronic study, the rats were given 200 mg/kg/day and 40 mg/kg/day for three months. No mortality was reported and the appetite behaviour and appearance of rats was not affected. The authors concluded that, in total, the dose tolerated in rats, under these conditions, can be fixed at least 200 mg/kg/day, which represents 10 times of the daily dosage expected in humans. The authors also reported that the drug was locally well supported. The product does not seem to have a teratogenic effect, even at very high doses and on the three species studied: rats, mice and rabbits. In particular, the drug was found to be well supported by pregnant rats even at 1200 mg/kg/day. There was also no effect on the development of the young rats.

The current invention serves to address the problems of the prior art and provides a medicament for use in the treatment of conditions of the nervous system. In particular, the current invention provides a medicament for use in the treatment of spinal cord injury.

Adult central nervous system axons do not retain regenerative ability. Following injury the extracellular matrix plays diverse roles in potentiating regenerative failure. Chondroitin sulphate proteoglycans are a family of extracellular matrix molecules which are upregulated in the glial scar after spinal cord injury and inhibits neurite outgrowth thus regeneration. In addition, chondroitin sulphate proteoglycans are also present in a specific structure called PNNs limiting plasticity for potential functional recovery. Immunohistochemistry of the spinal cord showed that PNNs are down regulated around motor neurones, resulting from hyaluronan synthesis inhibited by hymecromone. In addition, we also observed that the staining intensity chondroitin sulphate proteoglycans is drastically reduced in the spinal cord.

### SUMMARY OF THE INVENTION

An aspect of the invention provides 4-methylumbelliferone (herein referred to as the “PNN inhibitor (PNNi) of the invention”), a derivative or salt thereof, for use in the treatment of a condition of the nervous system.

In an embodiment, the condition of the nervous system is one associated with the formation of lesions. In one embodiment, the lesion is a glial scar. In one embodiment, the lesion is a plaque resulting from an accumulation of toxic protein aggregations. One example is an amyloid scar.

In yet a further embodiment, the condition of the nervous system may be selected from the group comprising conditions caused by trauma, injury, infection, degeneration, structural defects, tumours, and blood flow disruption.

The condition may be selected from the group comprising stroke, transient ischemic attack, myelopathy, haemorrhage, meningitis, encephalitis, bell's palsy, brain or spinal tumour, Parkinson's disease, Huntington chorea, and Alzheimer disease. It may be cerebral palsy.

In an embodiment, the condition of the nervous system may be an injury to the nervous system. Preferably, the condition is a spinal cord injury.

An aspect of the invention provides 4-methylumbelliferone, a derivative or salt thereof, i.e. the PNN inhibitor (PNNi) of the invention, for use in the treatment of a lesion associated with a condition of the nervous system. The condition may be one as disclosed herein.

An aspect of the invention provides a method for treatment of a condition of the nervous system in a subject, the method comprising administration of 4-methylumbelliferone (herein referred to as the “PNN inhibitor (PNNi) of the invention”), a derivative or salt thereof, to said subject. The condition may be one as disclosed herein.

In a further aspect, the current invention provides a method for treatment of a lesion associated with a condition of the nervous system in a subject. The method comprising administration of 4-methylumbelliferone (herein referred to as the “PNN inhibitor (PNNi) of the invention”), a derivative or salt thereof, to said subject. The condition may be one as disclosed herein.

### Definitions

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

When used herein the terms “nervous system” or “human nervous system”, refer to the part of the body that coordinates actions and transmits signals or messages between parts of the body. The nervous system comprises the central nervous system, or CNS, (brain and spinal cord) and the peripheral nervous system, or PNS.

When used herein the term “condition of the nervous system” may be any disease, disorder or condition, that affects normal function of the nervous system. The condition may be an injury or damage. The condition may be selected from, but is not limited to, those caused by trauma, injury, infection, degeneration, structural defects, tumours, blood flow disruption, and autoimmune disorders. The condition may be selected from, but is not limited to, stroke, transient ischemic attack, myelopathy, haemorrhage, meningitis, encephalitis, bell's palsy, brain or spinal tumour, Parkinson's disease, multiple sclerosis, myotrophic lateral sclerosis (ALS), Huntington chorea, and Alzheimer disease.

When used herein the term “spinal cord injury” refers to any injury or damage to the spinal cord, or parts thereof, that causes changes in its function. It may be at any site or segment of the spinal cord and the damage may be at any level. There may be one or more sites of injury. The injury includes an injury below the conus involving the peripheral nerves. For the avoidance of doubt the term includes: open, closed and penetrating injuries to the spinal cord. This includes complete and incomplete lesions, partial and complete transection, central cord syndrome, Brown Sèquard syndrome, cauda equina syndrome, and myelopathy and radiculopathy of any degree or type.

As used herein, the term “condition” is used to define any abnormal condition that impairs physiological function and is associated with specific symptoms. The term is used broadly to encompass any disease, disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a condition or disease or removes (or lessens the impact of) its cause(s). In this case, the term treatment may also include enhancing recovery. In this case, the term is used synonymously with the term “therapy”. Additionally, the terms “treatment” or “treating” refers to an intervention (e.g. the administration of an agent to a subject) which prevents or delays the onset or progression of a disease or reduces (or eradicates) its incidence within a treated population. The term treatment is used synonymously with the term “prophylaxis”.

As used herein, “an effective amount” or “a therapeutically effective amount” of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

In the context of treatment and effective amounts as defined above, the term subject (which is to be read to include “individual”, “animal”, “patient” or “mammal” where context permits) defines any subject, particularly a mammalian subject, for whom treatment is indicated. In preferred embodiments, the subject is a human. In an embodiment, the subject is an adult. In an embodiment, the subject is a paediatric aged subject, i.e. 21 years or less. The subject may be of any gender.

When used herein, the term “composition” should be understood to mean something made by the hand of man, and not including naturally occurring compositions. Compositions may be formulated in unit dosage form, i.e., in the form of discrete portions containing a unit dose, or a multiple or sub-unit of a unit dose.

When used herein, the term “pharmaceutical composition” relates to the PNNi of the invention or the composition of the invention, admixed with one or more pharmaceutically acceptable carriers, diluents or excipients. Even though the PNNi of the invention can be administered alone, it will generally be administered in admixture with a pharmaceutical carrier, excipient or diluent, particularly for human therapy. The pharmaceutical compositions may be for human or animal usage in human and veterinary medicine. Examples of such suitable excipients for the various different forms of pharmaceutical compositions described herein may be found in the “Handbook of Pharmaceutical Excipients, 8th Edition, Edited by A Wade and P J Weller. In particular, American Pharmaceutical Review “Opportunities and Challenges in Biologic Drug Discovery (Hooven, 2017), formulations for topical delivery are described in Topical drug delivery formulations edited by David Osborne and Antonio Aman, Taylor & Francis, the complete contents of which are incorporated herein by reference.

Acceptable carriers or diluents for therapeutic use are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of phydroxybenzoic acid. Antioxidants and suspending agents may be also used.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans.

When used herein the term “derivative” refers to a compound derived from 4-methylumbelliferone that has been modified, e.g. by a chemical reaction but retains the ability to remove PNN, i.e. to treat a condition of the nervous system as described herein. Methods to determine PNN removal may be as described herein. The derivative may have, but not limited to, one or more alkyl, aryl, acyl, hydroxyl, hydroxymethyl, methoxy, methyl and/or sulfonyl substituents compared with 4-methylumbelliferone. The term may be used interchangeably with functional derivative. 4-methylumbelliferone derivatives are well known in the art. Examples can be found in US2019269647 or US2018201640 and all are incorporated herein by reference. All such derivatives are contemplated within the scope of this disclosure.

A derivative or pharmaceutically acceptable salt can be used for the treatment methods of the present disclosure. The PNN inhibitor or compound of the invention may be in the form of a salt, but those skilled in medicinal chemistry will appreciate that the choice of salt is not critical, and other pharmaceutically-acceptable salts can be prepared by well-known methods. The PNN inhibitor or compound of the invention may be in the form of a metabolite or a prodrug.

When used herein the term “lesion” refers to abnormal change in an organ or part thereof due to injury or disease. It may include a scar or a plaque.

The term “sustained release” is used in a conventional sense relating to a delivery system of a compound or active, which provides the gradual release of this compound or active during a period of time and preferably, although not necessarily, with relatively constant compound release levels over a period of time.

Hymecromone is a hyaluronan synthesis inhibitor. It is (4-methylumbelliferone) (Andreichenko et al., 2019). Our data showed that a ten-day non-invasive oral administration down-regulates both chondroitin sulphate and hyaluronan in the spinal cord. This puts hymecromone as a prime candidate for reducing inhibitory Chondroitin sulphate proteoglycans after spinal cord injury.

The involvement of PNNs in limiting plasticity is a physiological event. Activation of plasticity can be observed even when PNNs are removed in normal physiological conditions. Chronic phase spinal cord injury presents similar features of PNNs as in normal physiology and are likewise beneficially responsive.

“Alkyl” refers to straight chain or branched alkyl of the number of carbon atoms specified (e.g., C₁-C₄ alkyl), or any number within this range (methyl, ethyl, propyl, i-propyl, butyl, i-butyl, t-butyl, etc.). The Alkyl may be C₁-C₁₈, C₁-C₁₂, or C₁-C₆.

“Aryl”: Any univalent organic radical derived from an aromatic hydrocarbon by removing a hydrogen atom. For example, a simple aryl group is phenyl (with the chemical formula C6H5), a group derived from benzene.

“Acyl”: A group of atoms consisting of a carbonyl group bonded to a R group. An acyl group is a functional group with formula RCO— where R is an alkyl group bound to the carbon atom with a single bond. Typically the acyl group is attached to a larger molecule such that the carbon and oxygen atoms are joined by a double bond. Acyl groups are formed when one or more hydroxyl groups are removed from an oxoacid. Even though acyl groups are almost exclusively discussed in organic chemistry, they may be derived from inorganic compounds, such as phosphonic acid and sulfonic acid. Esters, ketones, aldehydes and amides all contain the acyl group. Specific examples include acetyl chloride (CH₃COCl) and benzoyl chloride (C₆H₅COCl).

### BRIEF DESCRIPTION OF THE FIGURES

The invention will be more clearly understood from the following description of an embodiment thereof, given byway of example only, with reference to the accompanying drawings, in which:

FIG. 1 illustrates (A-B) lack of aggrecan (ACAN) staining (PNN marker) in the PNNs and extracellular matrix, particularly in the ventral horn, observed after treatment with the PNNi of the invention; (C-D) PNNi treatment induced attenuation of hyaluronan binding protein (HABP) surrounding parvalbumin (PV)-positive neurones and in the extracellular matrix.

FIG. 2 illustrates a Basso, Beattie and Bresnahan (BBB) hindlimb locomotor open field test apparatus (Basso et al., 1995). (A) Flat open field apparatus, approximately 1 m in diameter. (B) Rats are placed in the open field apparatus for 4 minutes, weekly, to be assessed for hindlimb locomotor performance.

FIG. 3 illustrates an apparatus for mechanical sensory assessment; the von Frey assay. Animals were placed into the complete base assembly for plantar stimulation (A) with a wire mesh bottom (B) and acclimatised for approximately 20 minutes. Von Frey hairs of increasing logarithmic thickness (C) were pushed through the wire mess bottom and perpendicularly depressed against the plantar surface of the left or right hindlimb, using the Dixon up-down method to determine the 50% withdrawal threshold.

FIG. 4 illustrates the timeline of the study of example illustrated by FIGS. 2 to 5.

FIG. 5 illustrates: (A) contusion force analysis in rats. All rats received a similar contusion strength in all experimental groups; (B) Basso, Beattie and Brenahan (BBB) scores obtained. The results demonstrated a better functional recovery in PNNi treated rats (daily treatment) after a moderate T9 contusion injury. While vehicle treated rats reached ˜10 scores 5 weeks after injury, the 4-MU treated group reached ˜15 scores; (C) Von Frey hair test results. Rats from different treatment groups did not show any difference in tactile sensitivity using von Frey hair test. All groups n=11.

FIG. 6 illustrates the daily dose calculations for the PNNi of the invention.

FIG. 7 illustrates perineuronal nets (PNNs) (A) A schematic diagram of PNNs (green) on the surface of neurones (B) Synaptic vesicles (red) are found clustered in the holes of the PNNs (green) (de Winter et al., 2016; Vo et al., 2013).

FIG. 8 is a representative image for the presence of PNNs on alpha motor neurones in the spinal cord (Galtrey et al., 2008; Irvine and Kwok, 2018). Aggrecan (ACAN) positive PNNs surround most alpha motor neurones (Mns) NeuN and ChAT co-localisation denotes Mns. (A) Percentage of Mns in the ventral motor pools surrounded by NeuN, ACAN-positive PNNs and their co-localisation (ACAN+/NeuN+). Confocal images showing ACAN-positive PNNs (B) surrounding NeuN-positive (C) and ChAT-positive Mns (D) in the spinal cord, respectively. Error bars±SD; n=3. Statistics one-way ANOVA; significance levels: * p<0.05. Scale bars, 100 μm.

FIG. 9 (A-S): Analysis of the efficacy of PNNi in removing PNNs in vitro (A-I). While untreated PNN⁺HEK cells illustrated a clear signal of WFA positive PNNs, (B), two days of PNNi treatment (0.5 mM or 1.0 mM) administered to PNN⁺HEK cells removed 86.4±4.47% (F_{3, 66}=73.60, p<0.0001 for all treated timepoints vs. untreated) of WFA-positive staining. The staining intensity is partially recovered to 43.6±14.6% of baseline lectin binding within three days post-treatment (p<0.0001 for 3 d and 5 d post-treatment vs. during, FIGS. C-I). Histology from animals terminated after ten days dosing revealed that both methods of PNNi administration were sufficient in decreasing WFA-positive binding throughout the CNS compared to non-treated animals (J-O). Quantification in the dorsal horn of the spinal cord illustrated that ten days of oral PNNi administration induced a partial removal of WFA-positive moieties to 71.0±7.20% of baseline ECM levels (t(3)=5.15, p=0.0142; FIG. 9P). Short-term PNNi was sufficient to induce changes to sensory but not motor functions in the treated intact rats (Q-S).

FIG. 10 (A-S): Perineuronal net inhibitor (PNNi) induces reorganisation of the sensorimotor map (M1) in intact rats. Using intracortical microstimulation (ICMS), mapping of the HL and FL cortical movement representations were used to investigate the functional organisation of the M1 of intact/sham animals after long-term treatment with the prospective plasticity enhancer, PNNi. ICMS was performed at stereotaxic coordinates within a right hemisphere craniotomy 5 mm rostral/caudal to bregma (B) (A) 11 weeks post mid-thoracic laminectomy (Sham) or for age-controlled intact rats. Individual ICMS maps were combined to give the representative heat map per group illustrating the percentage of animals for each stereotaxic coordinate where hindlimb (HL; B-D) or forelimb (FL; K-M) movements were able to be elicited. A baseline HL and FL cortical map was generated for Lister Hooded rats (B, K; dotted outlines in B-D, K-M) to compare the functional plasticity of groups with sham surgery (C, L) and with chronic PNNi administration (D, M). Measurements for HL analysis (F-G) are illustrated in E). H) Average area (mm²) of the HL representation reduced with PNNi treatment. PNNi treatment decreased the percentage of the intact HL intact epicentre that evoked HL (F) but not the percentage of total evoked HL area per group in the intact HL epicentre (G). Paired-pulse stimulation of field potentials for both (1) short interstimulus interval (20-40 ms) and (J) long (150-250 ms) interstimulus intervals revealed no alterations of short-term or long-term paired-pulse ratio observed between groups (I-J). Measurements for FL analysis (P-S) are illustrated in O). After PNNi treatment, FL movements were elicited in areas that are not associated with FL or HL movements (see right of the baseline HL map; white dotted outline K-M). Whilst the total surface area evoking FL did not significantly change (N), PNNi treatment in sham animals decreased the area that FL was evoked in the intact FL epicentre (P) and but did not cause a corresponding increase in FL movements evoked in the intact HL area (R). The percentage of total FL in the FL intact epicentre (Q) or HL intact epicentre (S) did not change with sham surgery or PNNi treatment. For all ICMS groups, Intact n=4, Sham n=4 and Sham/PNNi n=5. For paired pulse groups, Intact n=32, Sham n=15 and Sham/PNNi n=19. Statistics, one-way ANOVA; significance levels: *p<0.05 **p<0.01 ***p<0.001.

FIG. 11 (A-N): Forelimb (FL) shifts into hindlimb (HL) area of sensorimotor cortex (M1) after perineuronal net inhibitor (PNNi) and spinal cord injury (SCI) combination. PNNi and/or injury independently enhances cortical plasticity in spinal cord injured rats. Intracortical microstimulation (ICMS) was performed at stereotaxic coordinates within a right hemisphere craniotomy 5 mm rostral/caudal to bregma (B) (A) 11 weeks post-injury. Representative heat maps per group illustrate the percentage of animals for each stereotaxic coordinate where HL (B-C) or FL (D-E) movements were able to be elicited. The Lister Hooded rat baseline HL and FL cortical maps (dotted outlines in B-E) were compared to groups with mid-thoracic SCI (B, D) and/or long-term PNNi administration (C, E) to assess structural plasticity. HL movements were unable to be elicited after SCI (B-C). Measurements for FL analysis (K-N) are illustrated in J). After PNNi and/or SCI, FL movements were elicited in areas that previously elicited HL movements (see right of white dotted outline D-E). Whilst the total surface area evoking FL did not significantly change (F), nor the percentage of FL in the intact FL epicentre (K-L), there was an increase in FL area associated with the intact HL epicentre with PNNi treatment after injury, compared to sham control (p=0.197, M; p<0.05, N). G: Plot of the average field potential amplitudes vs. stimulus intensity per group. Synaptic responses were elicited in cortical layers II/III by electrical stimuli via bipolar electrodes placed in cortical layers V/VI. Recordings were performed on cortical slice at the following coordinates: ML: 2-3 mm and AP 1.40-1.8 mm caudal to bregma. The combination of injury and PNNi treatment elevated synaptic transmission (G). Paired-pulse stimulation of field potentials for both short (20-40 ms) and long (150-250 ms) interstimulus intervals, revealed no alterations of long-term paired-pulse ratio (PPR) observed between groups (1). However, a lower short-term PPR was observed with injury alone (p=0.052; H). Statistics, one-way ANOVA; for groups, Sham n=4, Vehicle n=3 and PNNi n=3. G: Data are presented as mean±SEM, n represents cortical slices. Statistics: two-way repeated measures analysis of variance (ANOVA). H-I: Statistics, one-way ANOVA; for groups,

FIG. 12 (A-E): Limiting PNNi administration alongside sustained rehabilitation allows further hindlimb (HL) motor recovery. When PNNi treatment was terminated 2-3 weeks before the end of the experiments (8 weeks PNNi treatment) allowing for PNN reformation, a further HL improvement was observed with animals that had continued rehabilitative training (A). Bar graph showing the percentage of animals that were able to achieve forelimb-hindlimb (FL-HL) coordination at 9 weeks post-injury (WPI) at the end of PNNi administration, 10 and 12 WPI (B). Stacked bar graphs showing classification of (C) HL and (D) forelimb (FL) steps on the horizontal ladder 9 and 12 WPI, where a hit: score of 3-6, slip: 1-2 and miss: 0 on the classical ladder scoring system (Metz and Whishaw, 2009). E) Left-right (L-R) average 50% withdrawal threshold for the plantar hindpaws determined from von Frey assays performed at 9 and 12 WPI did not show hyperalgesia. Consolidation of PNNs after termination of PNNi treatment does not induce sensory changes. (For groups, n=10 and 9 for 8 week PNNi and 8 week PNNi+T, respectively. Statistics, A, C-E: two-way mixed factorial ANOVA; significance levels: *p<0.05 **p<0.01 and ***p<0.001. A, C, D: Error bars are ±SEM.)

FIG. 13 (A-K): 8 week PNNi with sustained rehabilitation partially recovers cortical reorganisation of FL areas to intact organisation. Individual ICMS maps were combined to give the representative heat maps for each group showing the percentage of animals for each stereotaxic coordinate where no hindlimb (HL; B-C) but forelimb (FL; D-E) movements were able to be elicited. Dotted outlines denote intact baseline area for HL (B-C) and FL (D-E) to compare the functional plasticity of groups with 8 weeks PNNi administration (B, D) and 8 weeks PNNi with sustained treadmill training (C, E). F) Average area (mm²) of the FL representation reduced to normal/intact levels with limited PNNi treatment with sustained training only. Measurements for analysis (G-H, J-K) are illustrated in I). After spinal cord injury, FL movements were elicited in areas that previously elicited HL movements (baseline HL map shown in white dotted outline D-E). G) FL movements were mostly able to be evoked within the intact FL area, with a slight trend of a reduction with 8 week PNNi+T (p=0.243). H) The proportion of the total FL area evoked in the intact FL area decreased with 8 week PNNi treatment, with only a trend with sustained training (p=0.112). J) Following injury, FL movements could be elicited in the HL area with 8 week PNNi treatment. However, only with sustained training, a partial retraction of FL from the HL area was observed (p=0.514). K) The proportion of FL area evoked in the intact HL area appears to increase in comparison to uninjured control (p=0.114 8-week PNNi and p=0.177 week PNNi+T).

FIG. 14: JD009 and JD013 attenuate PNN formation. Efficacy of PNNi, JD009, and JD013 in reducing PNN formation were analysed using immunocytochemistry. Staining intensity was measured using the N-acetylgalactosamine-binding lectin WFA to label PNNs. PNNi at 1 mM and 2 mM was insufficient to cause substantial changes in PNN morphology and expression in cells in comparison to untreated cells. In contrast, both JD009 and JD013 treatments altered PNN expression in cells at 0.5 mM and 1 mM concentrations.

FIG. 15. Chronic PNNi treatment preferentially reduces expression of perineuronal nets (PNNs), labelled by key PNN components, in the ventral horn (VH). Rat spinal cord sections (T4-6) obtained at 12 weeks post-injury were stained and intensity was analysed in the VH. PNNi partially decreases number of PNNs, as labelled by aggrecan (ACAN; M) and Wisteria floribunda agglutinin (WFA; N), particularly after injury. Confocal images showing global ACAN (A-F) and WFA (G-L) expression in the VH with injury (A-C, G-I). PNNi-treated sham (D, J) animals show change in overall expression of PNNs. Whereas, injured animals, treated with PNNi alone (E, K) or with combination treatment of PNNi and training (F, L), showed reductions in WFA expression in the VH. Scale bars, 100 μm. For all graphs, error bars±SD; n=3 per treatment group. Statistics, one-way ANOVA; significance levels: * p<0.05 ** p<0.01 *** p<0.001.

### DETAILED DESCRIPTION OF THE INVENTION

The current inventors have surprisingly found that 4-methylumbelliferone (herein referred to as “PNN inhibitor (PNNi) of the invention”) can be used to treat conditions of the nervous system.

4-methylumbelliferone is a small molecule and has the following chemical structure:

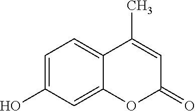

A derivative of 4-methylumbelliferone can be used for the use and treatment methods of the present invention. The derivative of 4-methylumbelliferone may be a modified form of 4-methylumbelliferone.

In an embodiment, the derivative of 4-methylumbelliferone is a compound of the following structure:

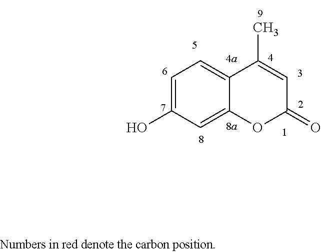

wherein, one or more alkyl, aryl, acyl, dimethyamino, hydroxyl, hydroxymethyl, methoxy, methyl, morpholino and sulfonyl substituents can be added. The substitution can take place at any position.

The substitution may be at position C₄. The substitution may be one or more of alkyl, aryl, acyl, dimethyamino, hydroxyl, hydroxymethyl, methoxy, methyl, morpholino and sulfonyl substituents. In one embodiment, NR₁R₂ can be added to the methyl group (CH₃) at C₄, wherein R₁ and/or R₂ can each independently be H, alkyl, aryl, acyl, sulfonyl. Preferably R₁ and R₂ are alkyl. The alkyl may be C₁ to C₁₈, for example C₁ to C₆.

Such a derivative, has the following structure:

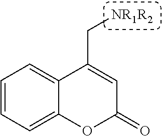

In one embodiment OR₁ can be added to the methyl group (CH₃) at C₄, wherein R₁ can be alkyl, aryl, acyl. Preferably R¹ is hydroxyethyl.

Such a derivative, has the following structure:

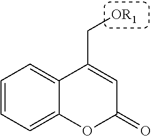

The substitution may be at position C3. The substitution may be one or more of alkyl, aryl, acyl, dimethyamino, hydroxyl, hydroxymethyl, methoxy, methyl, morpholino and sulfonyl substituents.

The substitution may be at position C5. The substitution may be one or more of alkyl, aryl, acyl, dimethyamino, hydroxyl, hydroxymethyl, methoxy, methyl, morpholino and sulfonyl substituents.

The substitution may be at position C6. The substitution may be one or more of alkyl, aryl, acyl, dimethyamino, hydroxyl, hydroxymethyl, methoxy, methyl, morpholino and sulfonyl substituents.

The substitution may be at position C8. The substitution may be one or more of alkyl, aryl, acyl, dimethyamino, hydroxyl, hydroxymethyl, methoxy, methyl, morpholino and sulfonyl substituents.

In one embodiment, the substitution may be at position C1. The substitution may be one or more of alkyl, aryl, acyl, dimethyamino, hydroxyl, hydroxymethyl, methoxy, methyl, morpholino and sulfonyl substituents. Preferably, the derivative is a compound of the following formula

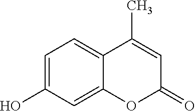

wherein O at position 1 is replaced with NR₁. R₁ can be aryl, acyl. Preferably R₁ is alkyl.

Notably, the derivative is a molecule or compound of the following structure in an embodiment of the invention.

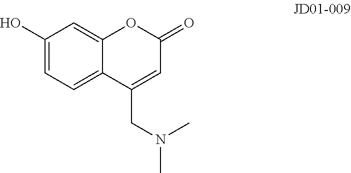 [SMILES: CN(C)Cc1cc(=O)oc2cc(O)ccc12]

### IUPAC Name: 4-[(dimethylamino)methyl-7-hydroxy-2H-1-benzopyran-2-one

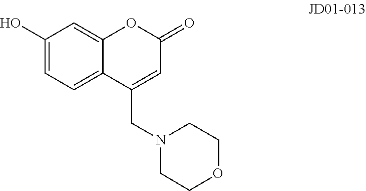 [SMILES: O=c1cc(CN2CCOCC2)c2ccc(O)cc2o1]

### IUPAC Name: 7-hydroxy-4-[(morpholin-4-yl)methyl]-2H-1-benzopyran-2-one

In an embodiment, the PNNi is hymecromone (C₁₀H₈O₃)

It will be appreciated that a pharmaceutically acceptable salt, metabolite or prodrug of 4-methylumbelliferone, or derivatives thereof, can be used for the use and treatment methods of the present invention. Features, uses and methods as disclosed herein in relation to the PNNi of the invention also apply to the derivatives, salts, metabolites and prodrug of 4-methylumbelliferone.

A further aspect of the invention provides a 4-methylumbelliferone derivative or salt thereof, and a composition comprising said derivative. The derivative is the derivative disclosed herein.

Perineuronal nets (PNNs) are dense pericellular extracellular matrix structures found throughout the central nervous system. PNNs wrap the surface of neurones. In particular, a population of neurones wrapped with PNNs are found in the spinal cord. PNNs surround most (˜97%) alpha motor neurones (Mns) in the spinal cord. The PNNs are aggrecan (ACAN)/CSPG-positive. After a spinal cord injury, for example, PNNs are degraded at the lesion site but remain intact in sites distant from the injury. Although PNNs are degraded at the lesion site, inhibitory CSPGs are up-regulated in the loose extracellular matrix.

The current inventors have surprisingly found that 4-methylumbelliferone, i.e. the PNNi of the invention, down-regulates hyaluronan and CPSGs, therefore removes PNNs in the central nervous system. Removal of PNNs opens a window of plasticity and promotes regeneration in the subject.

As shown in the accompanying examples, the PNNi of the invention induces attenuation of hyaluronan, as indicated by the reduction of hyaluronan binding protein (HABP) intensity, surrounding parvalbumin (PV)-positive neurones.

The current inventors have also found that the PNNi of the invention also functions to inhibit CSPGs synthesis in the central nervous system. Reducing CSPGs promotes plasticity.

In this regard, the PNNi of the invention can be used to treat conditions of the nervous system by promoting plasticity and regeneration. This action enhances recovery.

The condition of the nervous system may be a condition of the central or peripheral nervous system of a subject. The condition of the nervous system may be any condition associated with the formation of at least one lesion. The lesion is one with CSPG. The CSPG may be upregulated compared with a subject without the condition. The lesion may be a glial scar.

The lesion may be a plaque. The lesion may be in or near the spinal cord. The lesion may be in the brain.

The condition of the nervous system may be selected from the group comprising trauma, injury, infection, degeneration, structural defects, tumours, blood flow disruption, and autoimmune disorders. The condition may be selected from, but is not limited to, stroke, transient ischemic attach, haemorrhage, meningitis, encephalitis, bell's palsy, brain or spinal tumour, Parkinson's disease, multiple sclerosis, myotrophic lateral sclerosis (ALS), Huntington chorea, Alzheimer disease and cerebral palsy.

The condition of the nervous system may be a spinal cord injury. The injury may be at any segment of the spinal cord. It will be appreciated that the spinal cord injury may be any type of spinal cord injury and all are encompassed herein.

When used in the context of treatment of spinal cord injury, the PNNi of the invention and derivatives thereof, disrupt PNN formation and remove HA and CSPGs from PNNs and from the formed glial scar. This opens a window of plasticity and regeneration to promote recovery after spinal cord injury.

The PNNi of the invention may be administered at any time following spinal cord injury. It may be administered immediately after injury, within 1 hour after injury, within 2 to 12 hours after injury, or any time within the first seven days after injury. When the spinal cord injury is a chronic spinal cord injury administration may be at any time after injury. The subject could receive an initial administration of the PNNi of the invention, such as described above, and then optionally undergo long term administration. It can be continuous daily treatment or phasic treatments. Administration may be for any number of months or years, typically from about 1 month to about 36 months, or 6 months to 12 or 24 months. It will be understood that the pattern and period of administration will depend on the extent of the injury incurred.

The PNNi of the invention may be administered to a subject in combination with rehabilitation. The rehabilitation may take place before the PNNi of the invention is administered, during administration, i.e. concurrently, or after administration or any combination thereof. The PNNi of the invention may be the composition of the invention. Suitable methods of rehabilitation are known in the art and all are contemplated herein. Examples contemplated for use with the current invention include those disclosed in García-Alías et al. and Wang et al. (Garcia-Alias et al., 2009; Wang et al., 2011).

The PNNi of the invention may be administered in combination with electrostimulation. The electrostimulation may take place before the PNNi of the invention is administered, during administration, i.e. concurrently or after administration or any combination thereof. The PNNi of the invention may be the composition of the invention. Suitable methods of electrostimulation are known in the art and all are contemplated herein. Examples include those in U.S. 62/800,817 or U.S. Ser. No. 16/781,696.

The PNNi of the invention may be administered to a subject in combination with other treatments to maximise functional recovery. The other treatment may be a treatment for spinal cord injury. Such treatments are known in the art. The treatment may be an ISP peptide or a modified ISP peptide.

The PNNi of the invention may be a pharmaceutical composition. In this regard, the invention provides a pharmaceutical composition comprising a therapeutically effective amount of 4-methylumbelliferone, or a derivative thereof. The pharmaceutical composition is for use in the treatments as disclosed herein.

The invention also provides the PNNi of the invention for use in the treatment of a lesion of the nervous system. The lesion may be any lesion or scar in the central or peripheral nervous system of a subject.

In one embodiment, the lesion is a glial scar formed after spinal cord injury. In this regard, it will be appreciated that the invention provides a PNNi for use in the treatment of spinal cord injury. The treatment may be enhancing recovery after spinal cord injury. The lesion may be a glial scar. The lesion may be protein aggregated plaques. Other examples include one or more of amyloid lesion, tau aggregates and Lewy bodies.

Treatment of the lesion may be removal, completely or partially. Treatment may be breakdown of the lesion. Treatment may be such that normal function of the involved area or areas returns.

Methods of introduction or administration of the PNNi of the invention or the composition of the invention include but are not limited to intradermal, intramuscular, intraperitoneal, intravenous, subcutaneous, intranasal, epidural, intranasal, intracerebral, transrectal and oral routes. It may be by sublingual drop. The PNNi of the invention or the composition of the invention may be administered by any convenient route, for example by infusion or bolus injection, by absorption through epithelial or mucocutaneous linings (e.g., oral mucosa, rectal and intestinal mucosa, etc). Administration can be systemic or local. In addition, it may be desirable to introduce the formulation or composition of the invention into the central nervous system by any suitable route, including intraventricular and intrathecal injection; intraventricular injection may be facilitated by an intraventricular catheter, for example, attached to a reservoir. The PNNi or the composition of the invention may be formulated for slow release or sustained release.

The PNNi of the invention or composition may be formulated in accordance with routine procedures. The PNNi of the invention may be formulated in a formulation suitable for its administration. Typically, the PNNi of the invention or the composition of the invention may be formulated for oral delivery. The PNNi of the invention or the composition of the invention may be formulated for injection. In the context of spinal cord injury, injection may be directly into the spinal cord. Injection may be directly into the glial scar.

The PNNi of the invention or the composition of the invention may be formulated for release from a medical device. The medical device may be an implantable device, such as a patch or stent.

The PNNi of the invention and the composition comprising the PNNi of the invention may be prepared/formulated and/or administered in a variety of suitable forms. Such forms include, for example, but are not limited to, liquid, semi-solid and solid dosage forms, such as liquid solutions (e.g., injectable and infusible solutions), dispersions or suspensions, emulsions, microemulsions, tablets, pills, powders, liposomes, dendrimers and other nanoparticles, microparticles, and suppositories. It will be appreciated that the form may depend on the intended mode of administration.

In the context of spinal cord injury, the PNNi of the invention or the composition may be administered as an initial dose intravenously due to the condition of the subject. Subsequent doses may be given orally and/or intravenously. The method of administration is dependent on the condition of the patient, extent and/or location of the injury.

It will be appreciated that the dose of the PNNi of the invention depends on the condition and severity of the condition to be treated as well as the subject. It will depend on a variety of factors including the activity of the compound employed, the metabolic stability and length of action of that compound, the age, body weight, general health, sex, diet, mode and time of administration, rate of excretion, drug combination, the severity of the particular condition, and the individual undergoing therapy. There can, of course, be individual instances where higher or lower dosage ranges are merited, and such are within the scope of this invention. For example, the composition may be administered at a dose of from 5 to 60 mg/kg body weight/day, such as 10 to 50 mg/kg body weight, preferably 17 to 42 mg/kg body weight/day. In one embodiment from 1000 to 3000 mg/day, preferably from 1000 and 2000 mg/day is administered to the subject, preferably, 1200 to 1300 mg/day is administered to the subject. The amount and the frequency is as best suited to the purpose. The frequency of application or administration can vary greatly, depending on the needs of each subject, with a recommendation of an application or administration range from once a month to ten times a day, preferably from once a week to four times a day, more preferably from three times a week to three times a day, even more preferably once or twice a day. The length of treatment can vary greatly depending on the needs of the subject. In preferred embodiments, repeated use of is provided.

The dose may be one suitable for pediatric use.

In an embodiment, the PNNi is formulated in tablet form for oral administration. The tablet may comprise greater than 400 mg of the PNNi of the invention, preferably an amount from 500 mg to 600 mg. Each tablet is one dose and the frequency of the dose may be two times per day, or three times per day.

In an embodiment the subject may receive 2 to 3 doses per day, for example tables, for a period of months, such as 2 to 6 months, or 3 to 4 months, and optionally may have a period with no drug administration, e.g. 2 to 6 months, or 3 to 4 months. This regime may then be repeated. The dose may be as described herein.

### EXAMPLES

### Example 1

### Oral PNNi Administration Enhanced Functional Recovery of Spinal Cord Injury in the Spinal Cord of Animal Models of Acute Contusive Spinal Cord Injury

Methodology

Adult female Lister-hooded rats (200-250 g) were obtained from Charles River Laboratories (Canterbury, UK). Rats were housed in pairs in Central Biomedical Services (University of Leeds, UK) in a temperature-controlled environment in (20±1° C.), with a 12 hr light/dark cycle (lights on at 07:00). All procedures and experiments complied with the UK Animals (Scientific Procedures) Act 1986.

FIG. 4 outlines the timeline of the study.

Laminectomy and Contusion (Cx) Injury

Using isofluorane as an anaesthetic (5% in 02 for induction and 1-2% in 02 for the duration of the procedure), animals were shaved and sterilised. Vertebral segments T7-13 were exposed and a dorsal laminectomy was performed at T8. Vertebral levels T7 and T8 were stabilised whilst an Infinite Horizon impactor (Precision Systems and Instrumentation, LLC, Fairfax Station, Va.) was used to provide a 200 kdyn Cx (moderate injury) at the level of T9. Muscles were sutured and the skin was closed with autoclips. Analgesia (Vetagesic Buprenorphine; 0.015 mg/kg; Henry Schein Animal Health, Dumfries, UK) and antibiotics (Baytril enrofloxacin; 2.5 mg/kg; Henry Schein Animal Heath, Dumfries, UK) were given via subcutaneous injection immediately post-surgery and for three days following surgery.

Treatments

Following the surgical Cx injury, animals were grouped according to treatment paradigm to test the efficacy of the small molecule PNN inhibitor (PNNi), i.e. 4-methylumbelliferone, on enhancing recovery after SCI.

Due to the importance of rehabilitation as a SCI therapy, PNNi and rehabilitative training combination groups were included to look for issues of compatibility.

Pharmacological Administrations

Pharmacological treatment commenced from the day of injury (PNNi; 2 g/kg from a stock solution of 0.2 g/ml). This dosage is higher than the licensed dose of PNNi for treatment of a non-CNS-related disease and was established using preliminary in vitro experiments. Oral administration was achieved by syringe-feeding, twice daily to complete the daily dose, as opposed to via gavage.

As PNNi is an oral compound the length of administration can be controlled. Firstly, the drug was administered chronically from day of injury/surgery to the day of termination

Rehabilitation

Training was comprised of distributed practice quadrupedal interval treadmill training to provide task-specific rehabilitation. The first session commenced 7 days post injury (DPI) following locomotor behavioural tests described below. Daily training consisted of 10 minutes on the treadmill, followed by a 10 minute break before a final 10 minute session on the treadmill. Rats were trained five times a week at the maximal speed that they could maintain consecutive stepping for each 10-minute session on the treadmill.

Locomotor Assessments

Behavioural and functional assessments of hindlimb (HL) function were assessed throughout the study.

Basso, Beattie and Bresnahan (BBB) HL Locomotor Open Field Test

HL locomotor ability was assessed at various time points throughout the acute SCI paradigm using the BBB HL locomotor scale. BBB testing was carried out using an open locomotor field (custom-built Perspex O-ring: diameter 80 cm, height 30 cm) where animals were placed for a duration of 4 minutes (FIG. 2). Each BBB test was simultaneously assessed by two individuals. The resulting scores were pooled and averaged for objectivity. BBB testing assesses HL motor function using a ranking scale from 0-21. Animals are ranked into three broad categories based on their BBB score: early phase (score of 0-7) presenting little to no limb movement; intermediate stage (score of 8-13) with bouts of uncoordinated stepping; and the late stage (score of 14-21) presenting with FL and HL coordination and stability (Basso et al., 1995). Following injury, BBBs were then carried out at 1 DPI to confirm injury and then weekly from 7 DPI. If animals also received rehabilitative training, BBBs were carried out beforehand.

Von Frey Assay

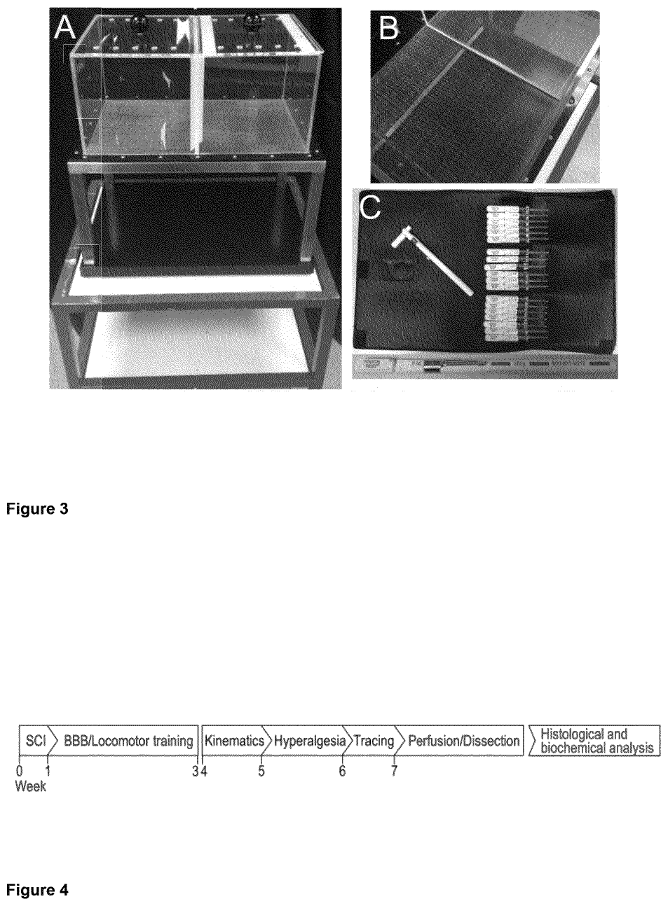

Changes to HL sensory function was assessed using von Frey methodologies to look for hyperalgesia and neuropathic pain. Four animals at a time were acclimatised to Perspex cages with a wire mesh bottom (FIG. 3) for approximately fifteen to twenty minutes before the test began and until general movement and grooming activities stopped. Von Frey filaments (Touch Test™ Sensory Evaluator Kit of 20; #39337500; Leica Biosystems, Milton Keynes, England FIG. 3C) were depressed through the mesh-bottomed cage against the more sensitive plantar arch of the HL footpads where withdrawal of the limb was counted as a positive result. Flinching was also seen as a positive response whereas walking was an ambiguous response requiring retesting after an appropriate delay. The left hindpaw and right hindpaw of all animals were performed in series to provide a sufficient interval between stimuli. Sensory testing procedure and analysis was carried out as described by Chaplin et al. (1994) (Chaplan et al., 1994) using the Dixon up-down method (Dixon, 1980) to determine the 50% withdrawal threshold for each HL.

Histology

Tissue Preparation

Animals were given an overdose of sodium pentobarbital (Pentoject; Henry Schein; 200 mg/kg; intraperitoneal injection) to deeply anaesthetise without halting cardiac function. A transcardial perfusion (Gage et al., 2012) was then performed using sodium phosphate buffer (PB; 0.12 M sodium phosphate monobasic; 0.1 M NaOH; pH 7.4) followed by 4% paraformaldehyde (PFA; in PB; pH 7.4) for tissue fixation. The brain and spinal cord were dissected out, post-fixed in PFA (4%; 4° C.) overnight and cryoprotected in 30% sucrose solution (30% v/w sucrose in PB; 4° C.) until tissue saturation. The left brain hemisphere and appropriate spinal cord segments were excised and frozen in optimum temperature medium (OCT; Leica FSC 22 Frozen Section Media; Leica Biosystems) before storage at −80° C. until sectioning. Sectioning of tissue was performed using a cryostat (Leica CM1850; Leica Biosystems) into 40 μm transverse sections for free-floating sections and collected into 48-well plates containing physiological buffer solution (PBS; 0.13 M sodium chloride, 0.7 M sodium phosphate dibasic, 0.003 M sodium phosphate monobasic; pH 7.4) to remove the OCT before being transferred to 30% sucrose solution for storage at 4° C.

Immunohistochemical Techniques

At room temperature (RT), sections were washed three times for 5 min each in Tris-buffered saline (TBS; 0.1 M tris base, 0.15 M NaCl; pH 7.4) to remove sucrose residue. Tissue was then blocked in 0.3% TBST (1×TBS solution and 0.3% v/v Triton X-100) and 3% normal donkey serum (NDS; v/v) for two hours. The sections were then transferred to co-incubate at 4° C. in blocking buffer (3% NDS in 0.3% TBST; pH 7.4) containing primary antibodies.

Following primary antibody or lectin incubation, sections were then washed thrice using TBS (10 mins; RT). To visualise each primary antibody staining, the tissue was then co-incubated in darkness with the fluorescent-conjugated secondary antibodies (1:500; 2 hrs; RT) against the species of the primary antibodies. Tissues were then washed three times in TBS (10 mins; RT) whilst protected from light. A final wash in Tris non-saline (TNS; 0.5 M Tris, pH 7.6) was given to reduce precipitation before air-drying. Tissues were mounted on Superfrost Plus slides, air-dried and coverslipped with the mounting medium FluorSave™ Reagent (EMD Millipore).

Primary antibodies are: CSPG components, including ACAN, BCAN and NCAN

Lectins: biotinylated Wisteria floribunda agglutinin (bio-WFA), biotinylated hyaluronan binding protein (bHABP).

Total number of neurones and the number of neurones surrounded by PNNs were quantified by two independent researchers, blinded to the study. Results were analysed with one-way ANOVA for statistical significance.

Determination of the Mechanism and Pharmacokinetics of PNNi

Aim (1) Mechanism of how PNNi Crosses Blood Brain Barrier (BBB)
Background: Our preliminary results have clearly demonstrated that an oral administration of PNNi leads to a reduction of PNNs in the central nervous system (CNS). However, the mechanism of how PNNi induces such effect remains not know. Does PNNi cross the BBB and deplete PNNi in situ? Or does PNNi deplete the pool of substrates systemically and thus less substrates would cross the BBB for PNN synthesis?
Methods: PNNi will be administered orally to adult rats for 10-consecutive days. Blood, urine and cerebrospinal fluid from the rats will be collected at day 0, 5 and 10 for the analysis of the concentration of PNNi present in the samples. The presence of PNNi will be measured using fluorescent spectrometry.
Animal numbers: 8 rats

Aim (2) Re-establish the pharmacokinetics of PNNi
Background: Previously published data on the pharmacokinetics indicated the lethal doses of oral administration in mouse and rat are 7.5 and 6.2 g/kg of weight of animals. However, in our pilot experiments in mice, we have administered between 6.8-12.8 g/kg in mice continuously for 6 months and yet the mice did not show any lethality and observe adverse signs. This prompts us to question if the previous pharmacokinetics were established correctly. Here, we aim to re-establish the pharmacokinetics of PNNi in adult rats and mice.
Methods: A thorough pharmacokinetic study will inevitably cover the doses leading to lethality of animals. Therefore, it is not possible to be performed this under any individual project license in the UK. We would there use the service from the company Charles River (a legitimate animal provider) to perform these experiments.

Related information can be found in this website:

https://www.criver.com/products-services/safety-assessment/dmpk/pharmacokinetics-toxicokinetics?region=3696

Results and Conclusion

Assessment of locomotor functions was performed weekly using Basso, Beattie, and Bresnahan (BBB). Nerve tracts were then traced both retro- and antero-gradely using cholera toxin B and biotinylated dextran.

Results from the BBB and Von Frey Test

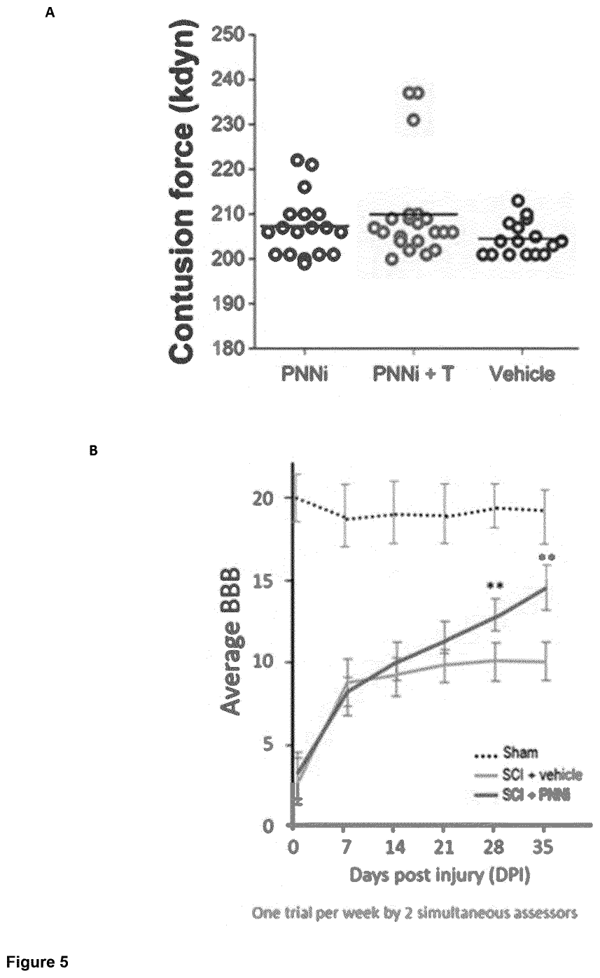

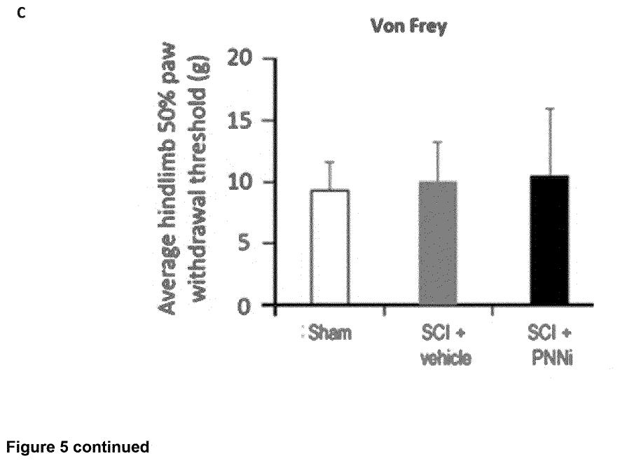

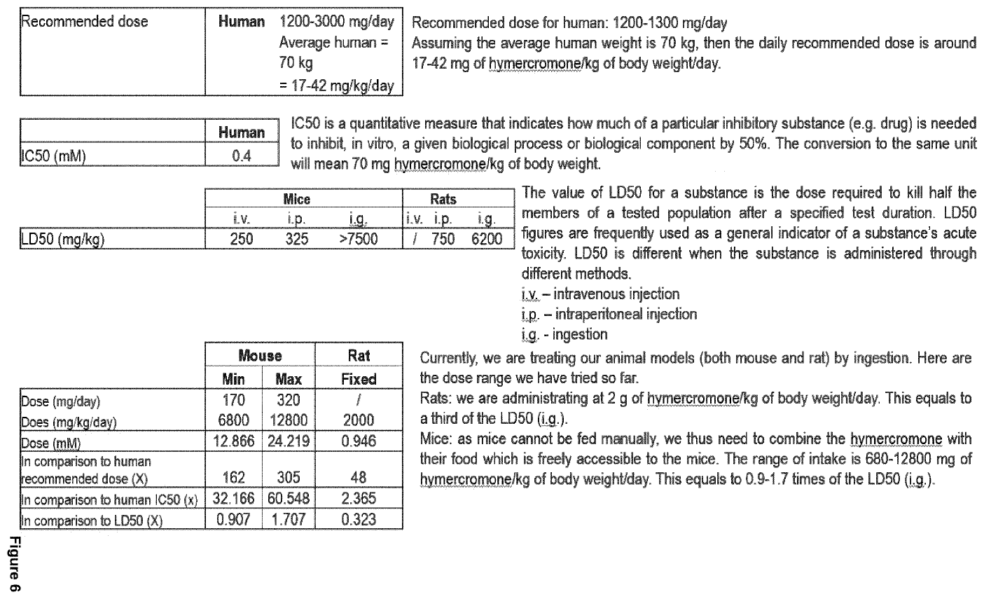

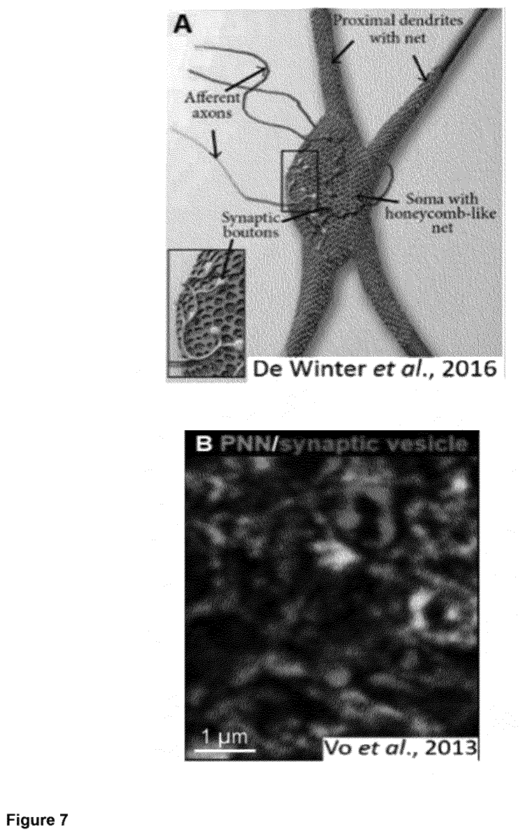

As illustrated in FIG. 5(A), all rats received a similar contusion strength in all experimental groups. (Middle) Basso, Beattie and Brenahan (BBB) scores demonstrated a better functional recovery in PNNi treated rats (daily treatment) after a moderate T9 contusion injury. While vehicle treated rats reached ˜10 scores 5 weeks after injury, the 4-MU treated group reached ˜15 scores. (Right) Rats from different treatment groups did not show any difference in tactile sensitivity using von Frey hair test. All groups n=11.

These results show that oral treatment using PNNi enhances functional recovery.

Immunochemical Results

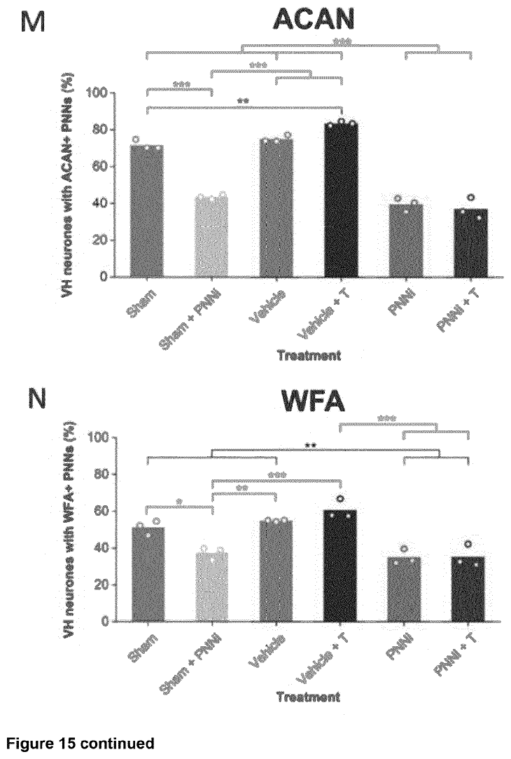

As illustrated in FIG. 15, chronic PNNi treatment reduces expression of PNNs, labelled by key PNN components, in the ventral horn (VH). Rat spinal cord sections (T4-6) obtained at 12 weeks post-injury were stained and intensity was analysed in the VH. PNNi partially decreases number of PNNs, as labelled by aggrecan (ACAN) and Wisteria floribunda agglutinin (WFA), particularly after injury.

### Example 2

### Removal of PNN by PNNi in Rats

Results and Conclusion

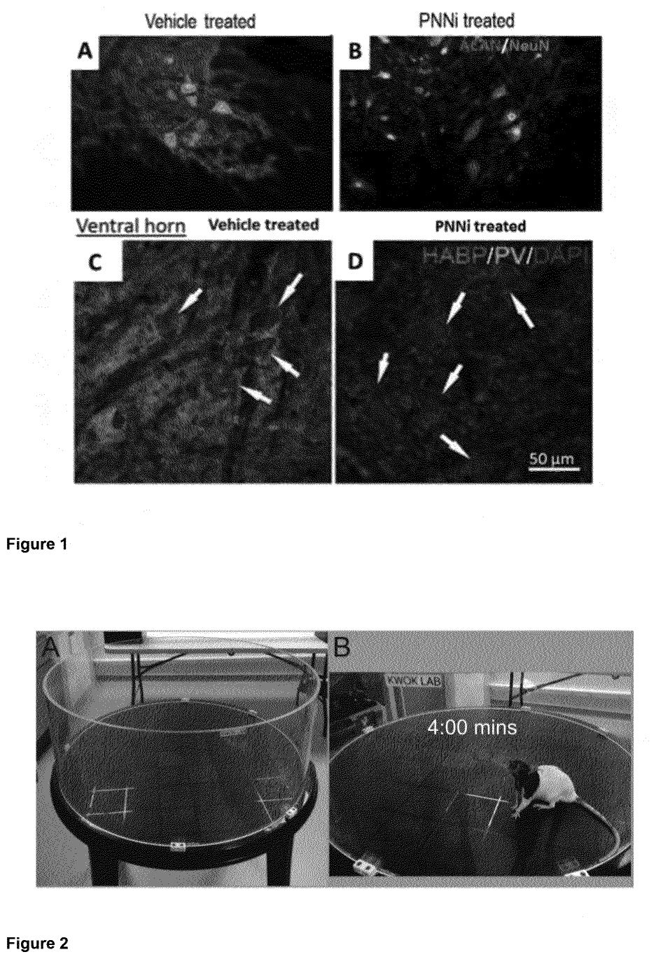

As illustrated in FIG. 1, after 10 days oral administration of PNNi in intact rats, PNNs are reduced. FIGS. 1 A and B shows lack of ACAN straining (CSPG; PNN marker), particularly in the ventral horn is observed after PNNi treatment. Figure C and D show that PNNi treatment induces attenuation of hyaluronan binding protein (HABP) surrounding parvalbumin (PV)-positive neurones.

This suggests that PNNi is also efficient in removing CSPGs in the spinal cord.

### Example 3

In this example, the current inventors introduce a non-invasive compound, PNN inhibitor (PNNi), to reversibly remove PNNs and enhance plasticity to remove PNNs via disruption of PNN formation to enhance recovery after acute spinal cord injury.

PNNs Surround Most (˜97%) Alpha Motor Neurones (Mns) in the Spinal Cord

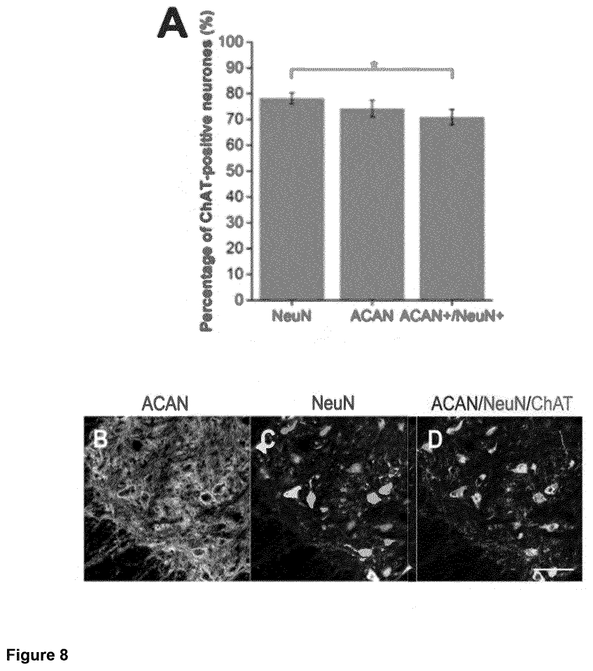

Most of the studies of PNNs are performed in brain samples, the inventors attempted to identify the population of neurones wrapped with PNNs in the spinal cord. FIG. 8 is a representative image for the presence of PNNs on alpha motor neurones in the spinal cord (Galtrey et al., 2008; Irvine and Kwok, 2018).

PNNi Dynamically Removes PNNs In Vitro and In Vivo

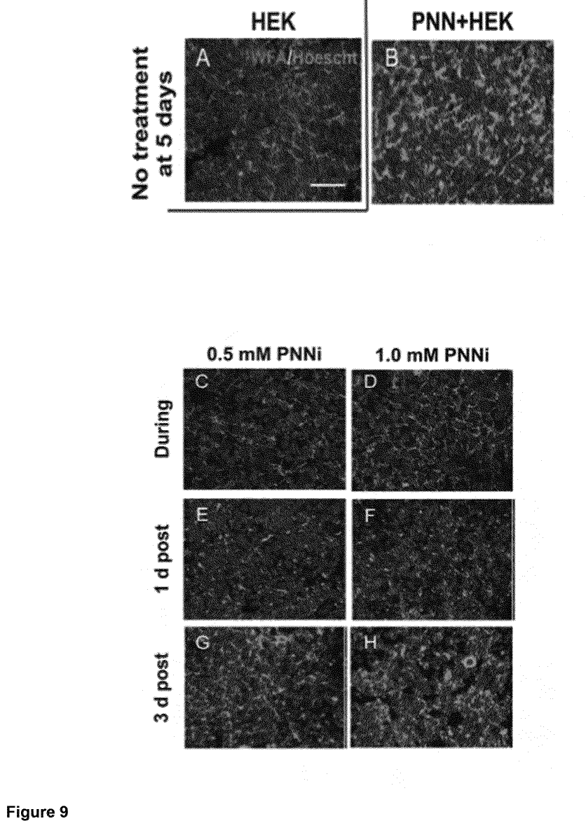

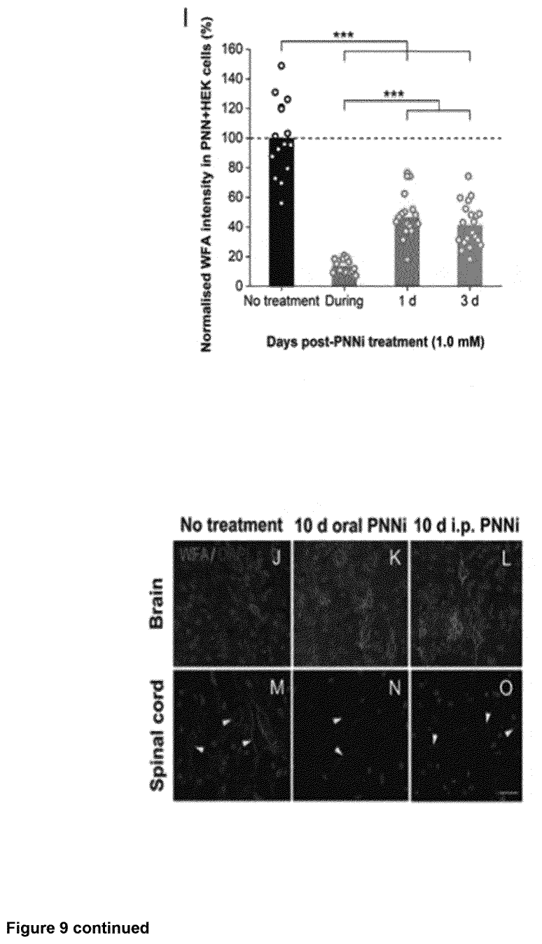

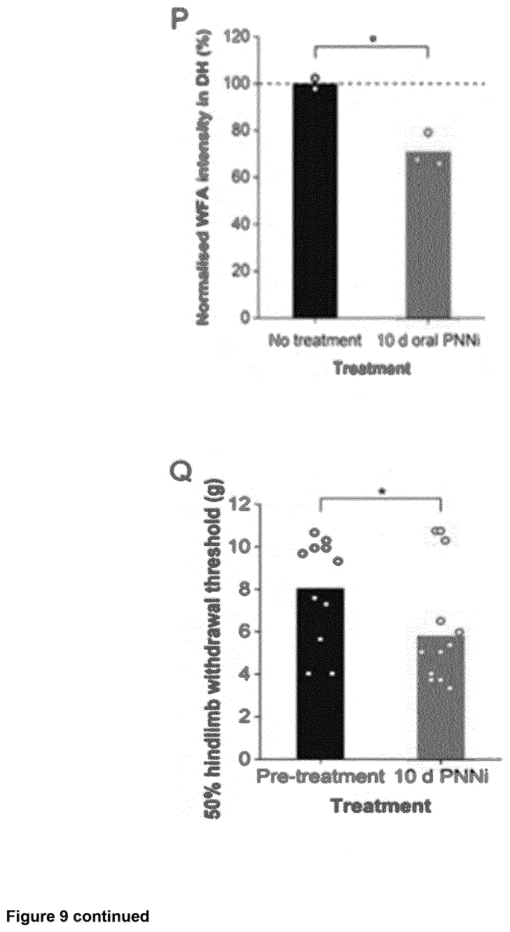

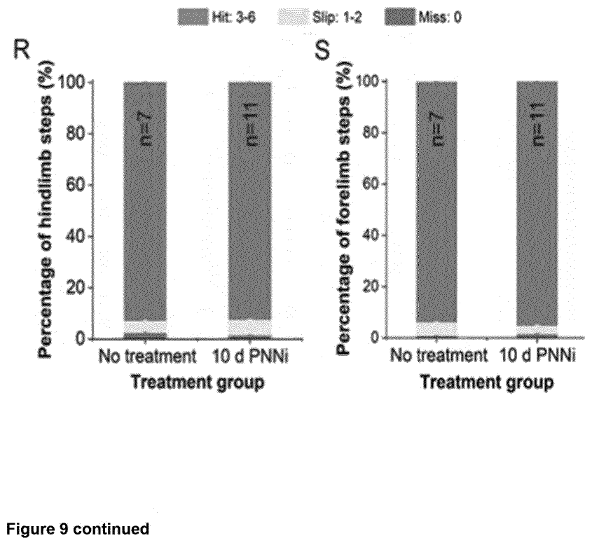

A previously developed human embryonic kidney 293T (HEK) cell model, engineered to express the essential ECM components (hyaluronan synthase 3 (HAS-3) and HAPLN-1) required to induce the formation of a pericellular PNN-like structure (PNN⁺HEK cells) (Kwok et al., 2010), was used to investigate the efficacy of PNNi in removing PNNs in vitro (FIG. 9A-I). While untreated PNN⁺HEK cells illustrated a clear signal of WFA positive PNNs, (FIG. 9B), two days of PNNi treatment (0.5 mM or 1.0 mM) administered to PNN⁺HEK cells removed 86.4±4.47% (F_{3, 66}=73.60, p<0.0001 for all treated timepoints vs. untreated) of WFA-positive staining. The staining intensity is partially recovered to 43.6±14.6% of baseline lectin binding within three days post-treatment (p<0.0001 for 3 d and 5 d post-treatment vs. during, FIG. 9C-1). These results indicate that PNNi-mediated removal of PNNs is dynamic and reversible.

Oral PNNi Administration Removes PNNs in the Spinal Cord

PNNi treatment was consequently investigated in vivo, with short-term administration (10 days) via either oral feeding or intraperitoneal (i.p.) injection twice daily. Histology from animals terminated after ten days dosing revealed that both methods of PNNi administration were sufficient in decreasing WFA-positive binding throughout the CNS compared to non-treated animals (FIG. 1J-O). Interestingly, PNNi appeared to be more efficacious in downregulating the lectin binding in the spinal cord in comparison to the cortex (FIG. 9J-O). Importantly, this indicates that PNNi, or its metabolites, can cross the blood brain barrier to affect the ECM in the CNS. Quantification in the dorsal horn of the spinal cord illustrated that ten days of oral PNNi administration induced a partial removal of WFA-positive moieties to 71.0±7.20% of baseline ECM levels (t(3)=5.15, p=0.0142; FIG. 9P). Short-term oral PNNi also downregulated staining with HA binding protein to 64.0±5.84% (HABP; t(3)=7.69, p=0.00456) and for HAPLN-1 to 68.3±8.09% (t(3)=5.01, p=0.0153) of baseline levels in the dorsal horn. As oral PNNi treatment is both non-invasive and generated sufficient effects on the neural ECM in vivo, the following PNNi dosing experiments with Lister Hooded rats used this method of administration.

Partial Removal of PNNs with Acute Short-Term Treatment of PNNi Showed No Adverse Effect

Firstly, the inventors questioned whether PNN removal throughout the CNS would affect normal sensation and motor functions. To investigate this, adult female Lister Hooded rats (n=11) were given ten days of oral PNNi treatment and subjected to routine behavioural testing. These revealed that short-term PNNi was sufficient to induce changes to sensory but not motor functions in the treated intact rats (FIG. 9Q-S). Comparison to a pre-treatment baseline (7.9±2.73 g) with the same rats, revealed that short-term PNNi administration decreased the withdrawal threshold to 5.5±2.03 g, representing an approximate 30% increase in sensitivity (t(10)=2.76; p=0.02 FIG. 9Q). Open field locomotor testing found no significant differences with short-term PNNi treatment, with all animals achieving top scores of 21 on the Basso, Beattie, Bresnahan hindlimb (HL) scale (Basso et al., 1995) (n.s.; p=1; data not shown). When a more skilled walking task was used to assess locomotion, HL locomotor activity also appeared consistent between PNNi treated and untreated animals (FIG. 9R), with correct stepping (green) approximately 92.7±0.82% (n.s., t(16)=0.259, p=0.799) of the time, with slips (yellow) and misses (red) making up 5.43±0.70% (n.s., t(16)=−0.978, p=0.343) and 1.92±0.41% (n.s., t(16)=1.15, p=0.268) of all steps, respectively, on the horizontal ladder. Similarly, forelimb (FL) performance was unaffected by short-term PNNi (FIG. 9S), with 1.13±0.24% missed (n.s., t(16)=−1.64, p=0.121), 4.35±0.54% slipped (n.s., t(16)=1.97, p=0.0667) and 94.4±0.56% correct steps (n.s., t(16)=−1.34, p=0.197).

Regardless of treatment, HL and FL function performed equally as well with low percentages of stepping errors. Overall, acute removal of PNNs in the normal adult rat slightly increased sensitivity in the limbs tested, aligning with downregulation of PNN components in the dorsal horn with the same treatment paradigm, but did not affect locomotor function. No other behavioural differences were observed.

The sensorimotor cortex (M1) contains a highly organised topographical representation of motor movements that is subject to structural and functional plasticity in response to sensorimotor learning or neuronal injury.

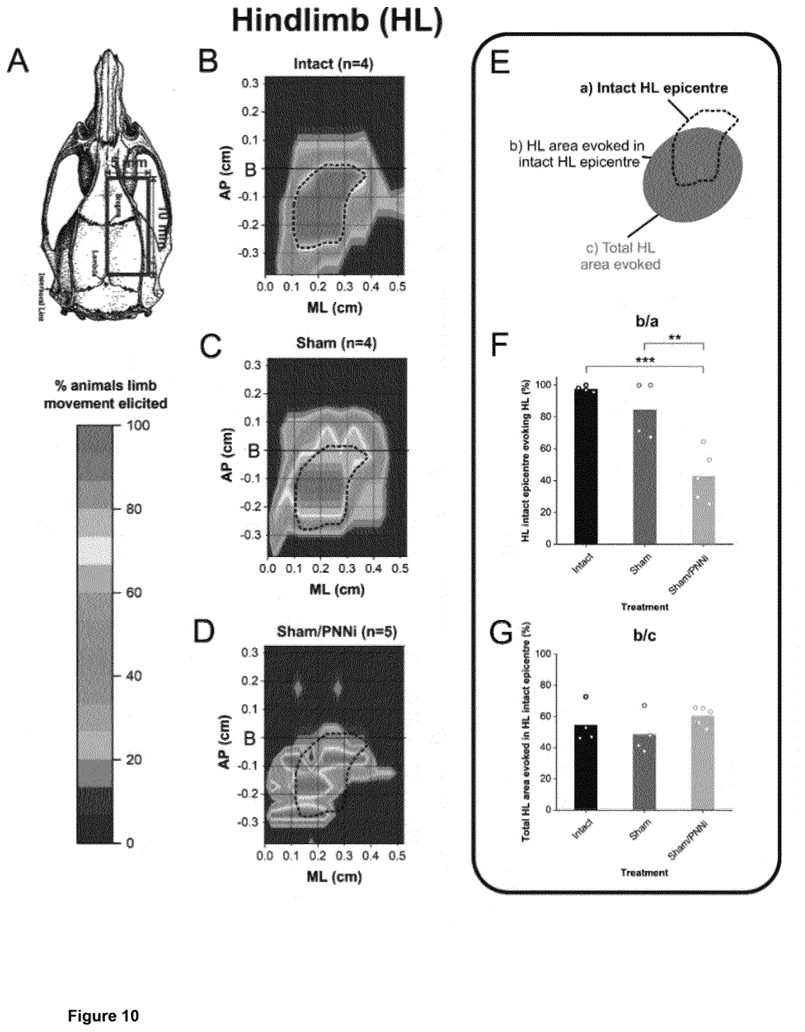

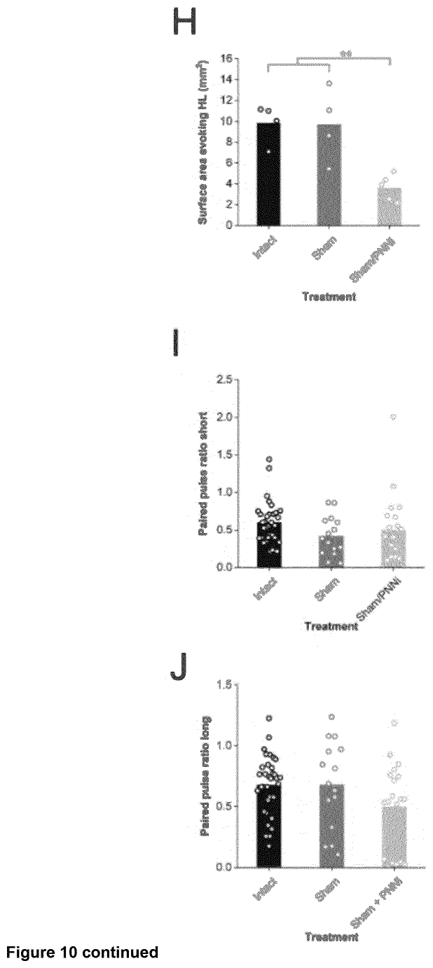

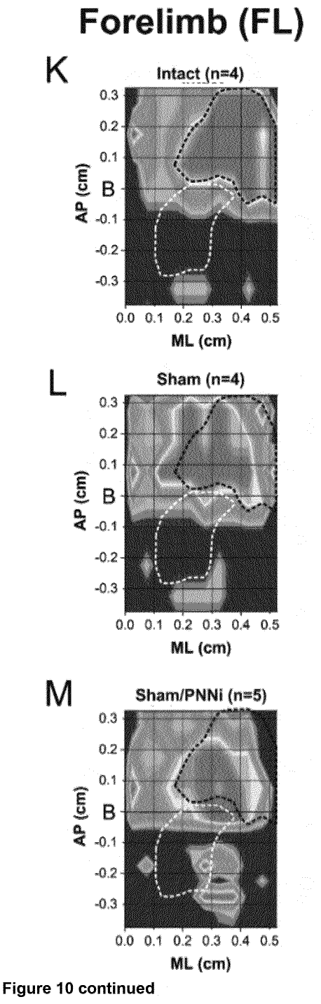

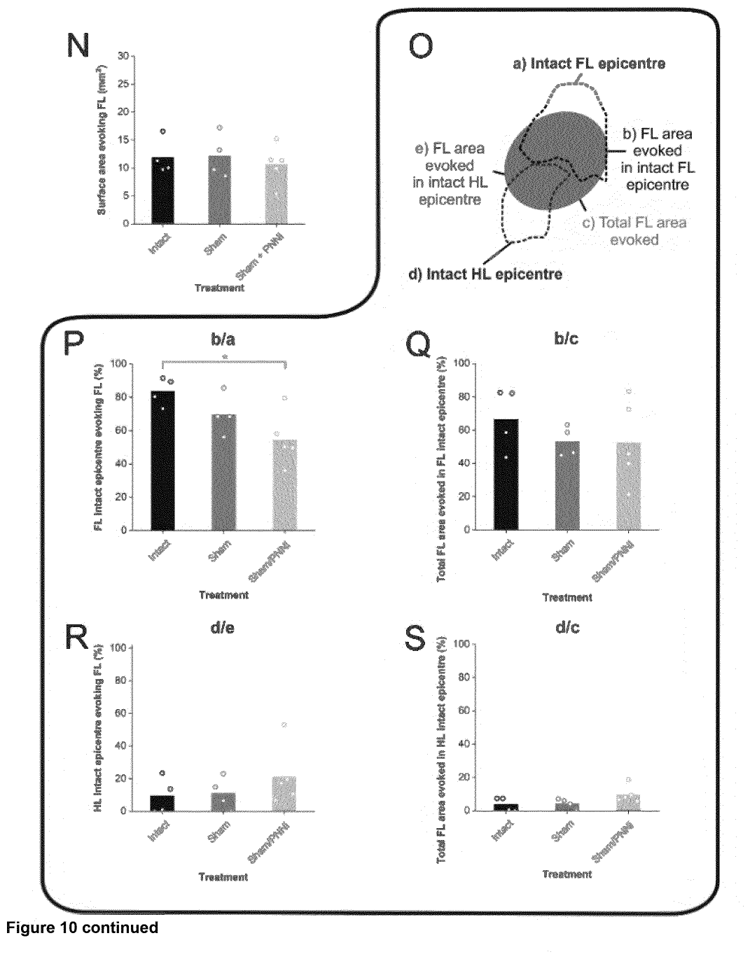

Using intracortical microstimulation (ICMS), mapping of the HL and FL cortical movement representations were used to investigate the functional organisation of the M1 of intact/sham animals after long-term treatment with the prospective plasticity enhancer, PNNi by comparing to the intact baseline HL and FL representations or ‘epicentres’ (see dotted lines; FIG. 10). Whilst PNNi treatment reduced the total area able to elicit HL movements (F_{2, 12}=10.7, p=0.00764 for Sham/PNNi vs. Intact, p=0.00914 for Sham/PNNi vs. Sham), this was due to some of the intact HL epicentre being no longer able to elicit HL movements (F_{2, 12}=18.6, p=0.00411 for Sham vs. Sham/PNNi, p<0.001 for Intact vs. Sham/PNNi; FIG. 10A-H). Paired-pulse protocols (Gigout et al., 2013; Luhmann et al., 1995) were used as an index to study possible alteration of intracortical GABAergic inhibition in parallel, using in vitro sensorimotor cortical slices, where the paired-pulse ratios (PPR) for short and long interstimulus intervals allowed us to investigate GABA_{A} and GABA_{B} receptor-mediated inhibition, respectively. Healthy control slices exhibited a pronounced paired-pulse inhibition (PPR: ˜0.5), however, this was not altered with either sham surgery or PNNi treatment (n.s., F_{2,65}=1.52, p=0.227, FIG. 10I; F_{2,65}=2.35, p=0.0960, FIG. 10J). This suggested that the observed PNNi-induced decreases in HL area was due to structural reorganisation and not alteration of cortical inhibition—an evidence of neural plasticity.

Like that observed with the HL representation, PNNi induced functional reorganisation of the FL representation in sham animals (FIG. 10K-S). Despite no change in the total surface area that elicited FL movements (n.s., F2, 12=0.249, p=0.784; FIG. 2N), the incidence of these were reduced in the intact FL epicentre (F2, 12=5.61, p=0.0226; FIG. 2P) but did not appear to encroach on the HL area (n.s., F2, 12=0.900, p=0.437, FIG. 2R; F2, 12=2.38, p=0.142, FIG. 10S). There was no significant change in cortical stimulation thresholds required to elicit HL or FL movements with PNNi treatment. As short-term PNNi administration increased tactile sensitivity and long-term PNNi treatment induced structural reorganisation, we can conclude that the partial removal of PNNs by PNNi, as illustrated above, was sufficient in producing an environment favourable to promoting functional plasticity at multiple levels of the CNS. As these plastic changes have been observed in treated intact animals, this may result in maladaptive connectivity.

PNNi and/or Injury Independently Enhances Cortical Plasticity in Spinal Cord Injured Rats

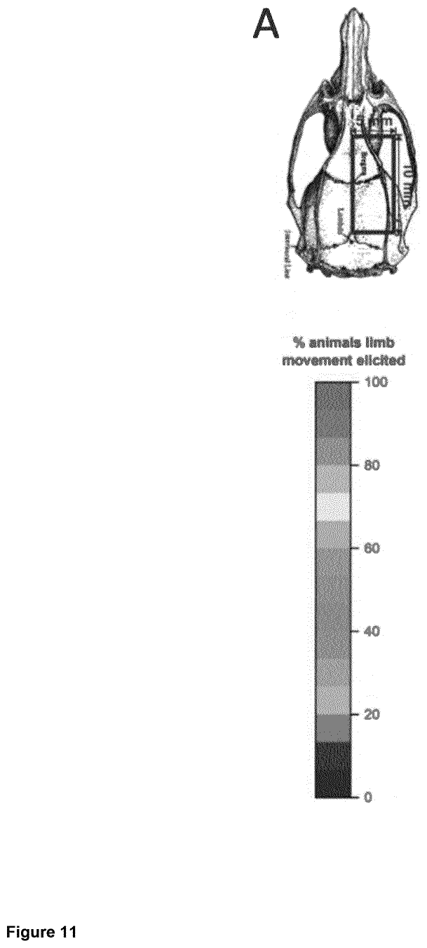

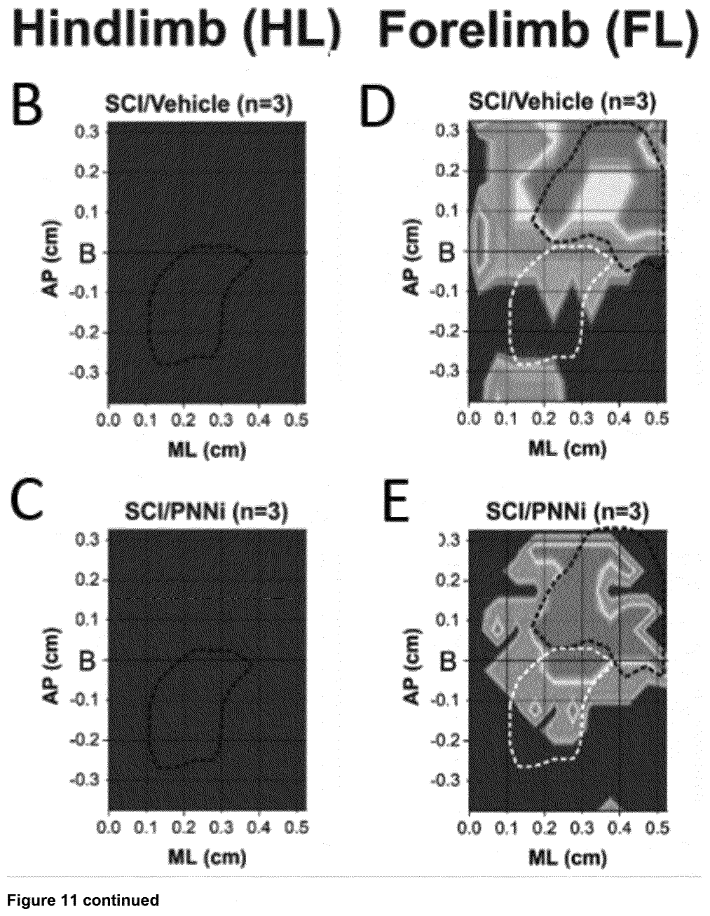

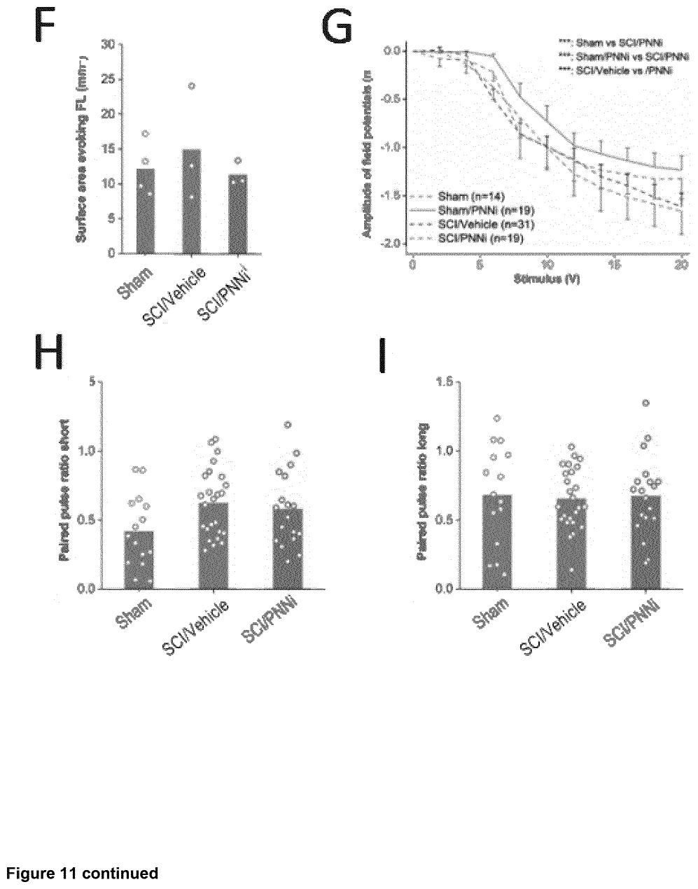

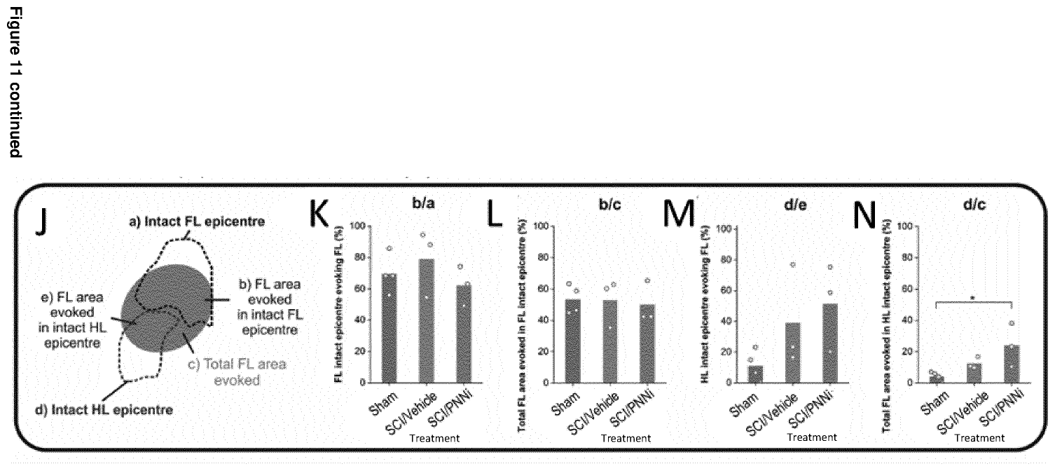

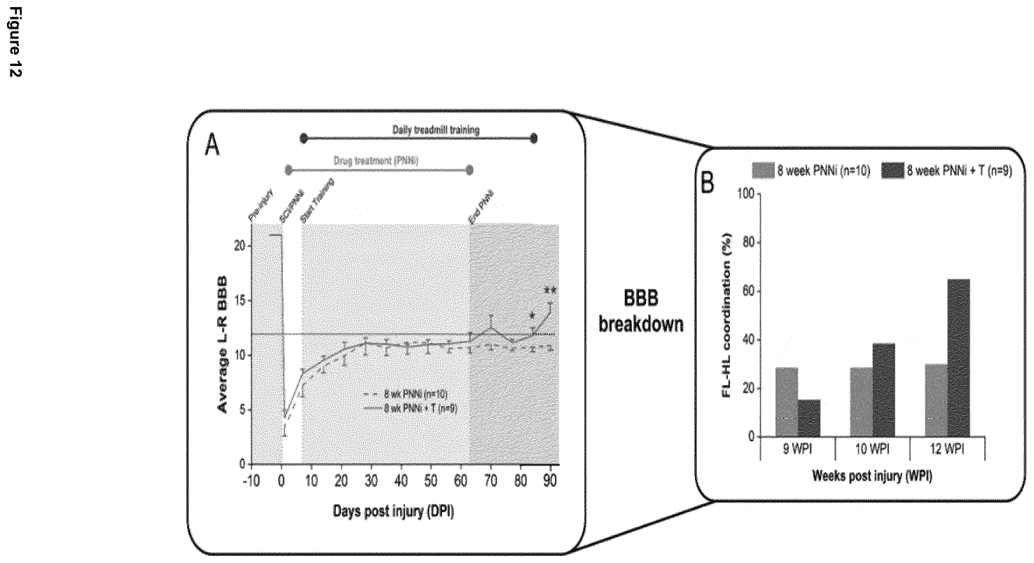

After mid-thoracic SCI, HL movements were unable to be elicited by ICMS (FIG. 11B-C), despite evidence of open-field HL ambulation, whereas FL movements were able to be elicited in both intact FL and HL areas (FIG. 11D-E). Injury and/or treatment caused no changes to the minimum or mean cortical stimulation thresholds required to elicit FL movements (Supplemental Table 1). There was no total change in the surface area eliciting FL movements with injury and/or long-term PNNi treatment (n.s., F_{2,9}=0.403, p=0.683, FIG. 5F), nor the area associated with the intact FL epicentre (n.s., F_{2,9}=0.891, p=0.452, FIG. 5K; F_{2,9}=0.0685, p=0.934, FIG. 5L). However, the combination of both injury and long-term PNNi treatment caused a larger percentage of FL movements (from 4.27±1.56% to 23.9±8.01%) to be associated with areas previously eliciting HL movements, indicating an overall shift, not expansion, of the group's representative FL area into the intact HL epicentre, compared to sham control (F_{2, 9}=2.58, p=0.197 for Sham vs. SCI/PNNi, FIG. 5M; F_{2, 9}=5.23, p=0.0432 for Sham vs. SCI/PNNi, FIG. 11N).

Parallel experiments addressing the local excitability of in vitro cortical slices, used individual input-output curves of field potential amplitudes (FIG. 11G) which were fitted by the Boltzmann equation, yielding the PSPₘₐₓ, the I₅₀ and the slope factor. Importantly, this revealed that the combination of SCI and PNNi treatment gave results significantly different from all other experimental groups (Greenhouse-Geisser F_{1.49, 13.4}=1.61, p<0.001 for all groups vs. SCI/PNNi, FIG. 11G), suggesting that together PNNi and injury created an environment of increased intracortical synaptic transmission, particularly at higher stimulation intensities, which may contribute to the reorganisation of the M1 observed with this group. Paired-pulse protocol also revealed that injury alone, not the SCI/PNNi combination, affected paired pulse responses at short but not long interstimulus intervals, with an increase in PPR from 0.42±0.26 to 0.63±0.24 (F_{2, 57}=3.09, p=0.052 for Sham vs. SCI/Vehicle, FIG. 5H-I), implying that SCI induced a decrease in GABA_{A}-receptor mediated transmission. In summary, despite the ability of both injury and PNNi to independently induce similar decreases in CSPG content in the M1, only long-term PNNi administration could enhance plasticity sufficiently enough to foster reorganisation of the cortical motor map, suggesting that additional mechanisms, such as the observed increase in local intracortical excitability, may underlie these functional changes.

Limiting PNNi Treatment Alongside Sustained Rehabilitation Allows Motor Recovery

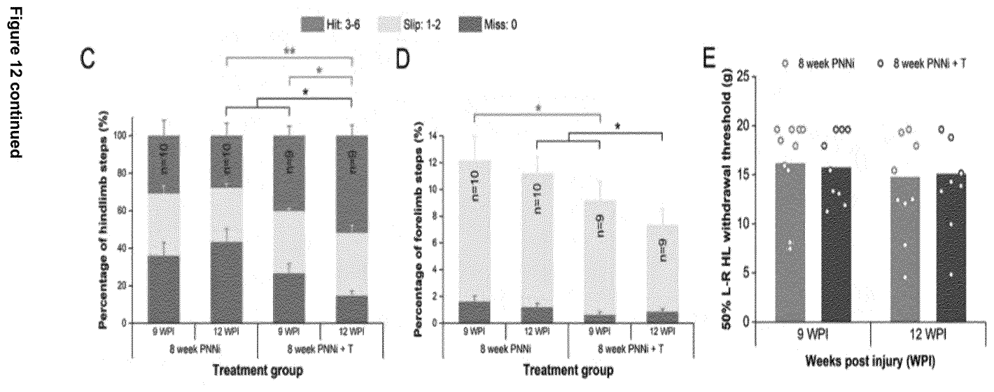

In order to enhance plasticity for effective functional recovery, we administer PNNi for 8 weeks in combination with 11 weeks of rehabilitation. Limiting PNNi administration alongside sustained rehabilitation allows further hindlimb (HL) motor but not sensory recovery (FIG. 12). When PNNi treatment was terminated 2-3 weeks before the end of the experiments (8 weeks PNNi treatment) allowing for PNN reformation, a further HL improvement was observed with animals that had continued rehabilitative training (A). Bar graph showing the percentage of animals that were able to achieve forelimb-hindlimb (FL-HL) coordination at 9 weeks post-injury (WPI) at the end of PNNi administration and 12 WPI (B). Animals that sustained further rehabilitation training (8 week PNNi+T) showed an increase in percentage able to FL-HL coordinate from 9 WPI to 12 WPI (A and B). Stacked bar graphs showing classification of (C) HL and (D) forelimb (FL) steps on the horizontal ladder 9 and 12 WPI, where a hit: score of 3-6, slip: 1-2 and miss: 0 on the classical ladder scoring system (Metz and Whishaw, 2009). HL performance on the ladder improved when PNNi treatment was limited and only with sustained rehabilitation treatment. Whilst the total error reduced (black), the limited PNNi treatment and rehabilitative training specifically decreased the number of missed HL steps (red). FL performance also improved with limited PNNi treatment with sustained training. E) Left-right (L-R) average 50% withdrawal threshold for the plantar hindpaws determined from von Frey assays performed at 9 and 12 WPI did not show hyperalgesia. Consolidation of PNNs after termination of PNNi treatment does not induce sensory changes. (For groups, n=10 and 9 for 8 week PNNi and 8 week PNNi+T, respectively. Statistics, A, C-E: two-way mixed factorial ANOVA; significance levels: *p<0.05 **p<0.01 and ***p<0.001. A, C, D: Error bars are ±SEM.)

8 Week PNNi with Sustained Rehabilitation Partially Recovers Cortical Reorganisation of FL Areas to Intact Organisation.

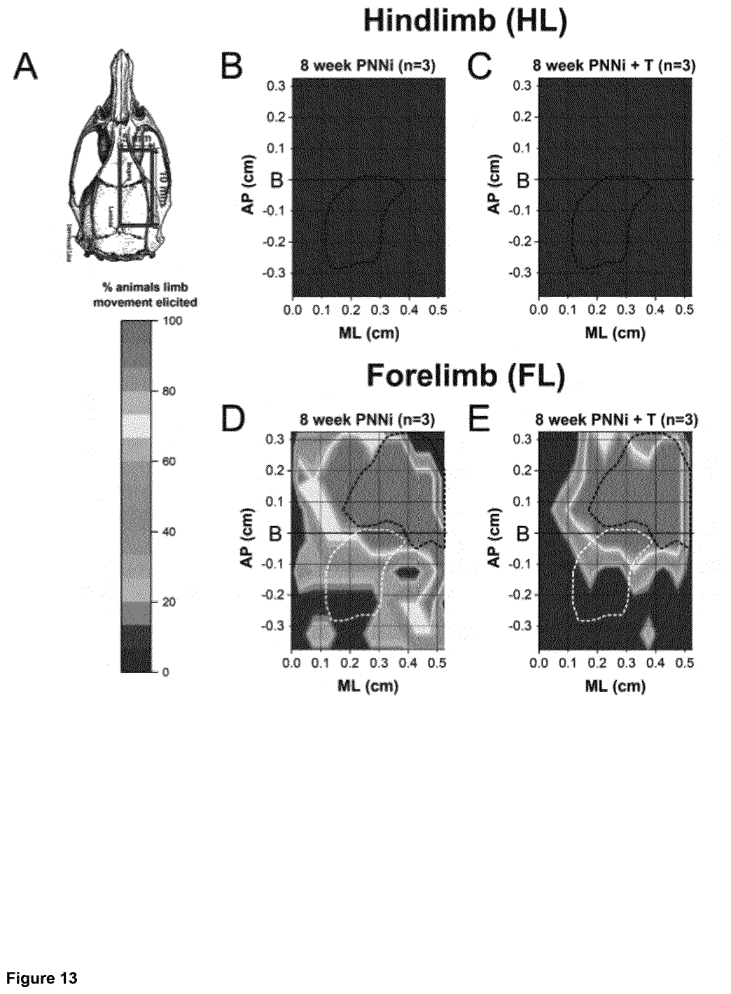

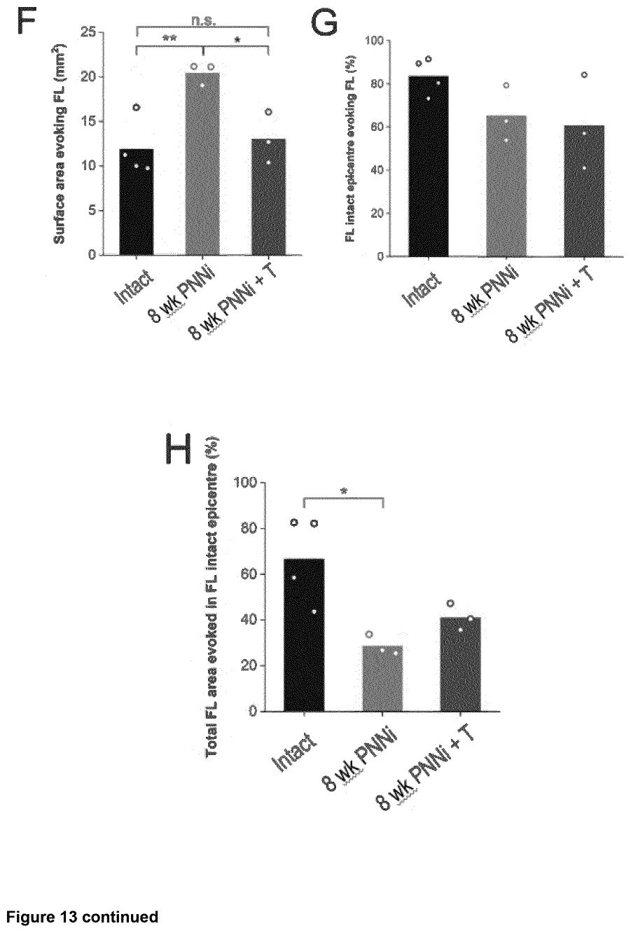

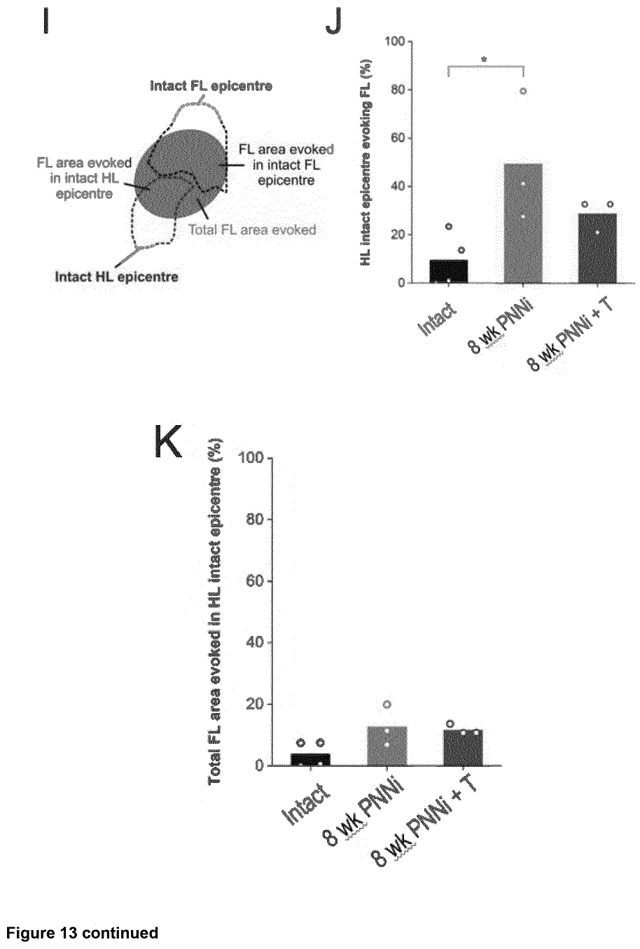

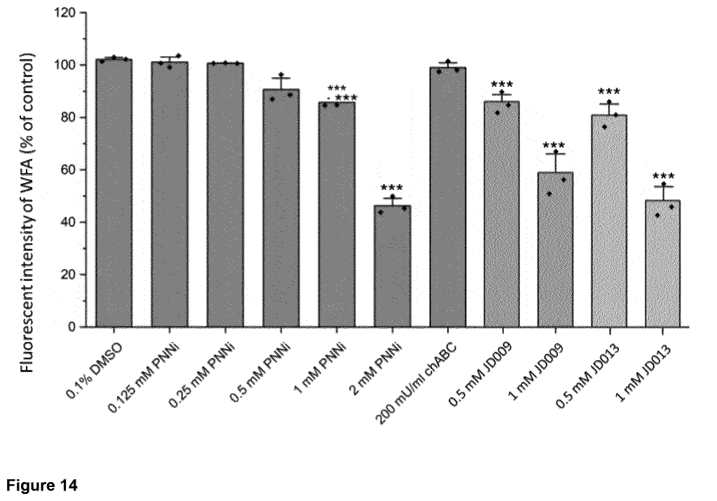

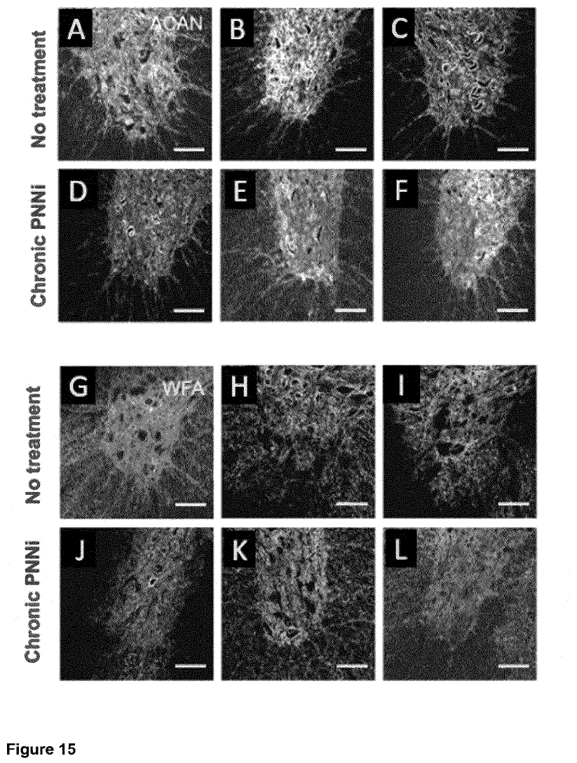

Intracortical microstimulation (ICMS) was performed at stereotaxic coordinates within a craniotomy 5 mm above and below bregma (labelled B on each scale) on the right hemisphere (A) approximately 15 weeks after a mid-thoracic moderate contusion injury (FIG. 13). Individual ICMS maps were combined to give the representative heat maps for each group showing the percentage of animals for each stereotaxic coordinate where no hindlimb (HL; B-C) but forelimb (FL; D-E) movements were able to be elicited. Dotted outlines denote intact baseline area for HL (B-C) and FL (D-E) to compare the functional plasticity of groups with 8 weeks PNNi administration (B, D) and 8 weeks PNNi with sustained treadmill training (C, E). F) Average area (mm²) of the FL representation reduced to normal/intact levels with limited PNNi treatment with sustained training only. Measurements for analysis (G-H, J-K) are illustrated in I). After spinal cord injury, FL movements were elicited in areas that previously elicited HL movements (baseline HL map shown in white dotted outline D-E). G) FL movements were mostly able to be evoked within the intact FL area, with a slight trend of a reduction with 8 week PNNi+T (p=0.243). H) The proportion of the total FL area evoked in the intact FL area decreased with 8 week PNNi treatment, with only a trend with sustained training (p=0.112). J) Following injury, FL movements could be elicited in the HL area with 8 week PNNi treatment. However, only with sustained training, a partial retraction of FL from the HL area was observed (p=0.514). K) The proportion of FL area evoked in the intact HL area appears to increase in comparison to uninjured control (p=0.114 8 week PNNi and p=0.177 week PNNi+T).

This suggests that limited PNNi combined with sustained rehabilitation limit maladaptive plasticity and consolidate the newly established connections from rehabilitation for functional recovery.

Preliminary Results of PNN Analogues

JD009 and JD013 May Attenuate PNN Formation at Lower Concentrations than PNNi

Given that PNNi is near insoluble in aqueous solution (Nagy et al., 2015), derivatives of PNNi with increased solubility (JD009 and JD013) were developed. The inventors hypothesised that since the hydroxyl functional groups required for activity were not altered when designing the PNNi analogues, they should retain the capacity to potently reduce PNNs.

To compare the efficacy of PNNi, JD009, and JD013 in reducing PNN formation, immunocytochemistry was conducted using the N-acetylgalactosamine-binding lectin WFA to label PNNs. PNN-HEK293 cells were exposed to 0.5 mM and 1 mM of the compounds for 3 days in vitro. DMSO vehicle control was also included, and the amount used did not exceed 0.1% of the total volume. Quantification of WFA fluorescence intensity per cell was derived in matched regions of interest (ROI) and normalising overall WFA fluorescence intensity to cell number. The figure below showed that 0.5 and 1 mM doses of PNNi were insufficient to cause substantial changes in PNN morphology and expression in cells in comparison to untreated cells. In contrast, both JD009 and JD013 treatments altered PNN expression in cells at 0.5 mM and 1 mM concentrations.

### REFERENCES

24 Moshayedi P, Ng G, Kwok J C F, Yeo G S H, Bryant C E, Fawcett J W, Franze K, Guck J (2014) The relationship between glial cell mechanosensitivity and foreign body reactions in the central nervous system. Biomaterials 35, 3919-25. (106 citations)

2013
23^{§} Dick G, Tan C T, Alves J N, Ehlert E M E, Miller G M, Hsieh-Wilson L C, Sugahara K, Oosterhof A, van Kuppevelt T H, Verhaagen J, Fawcett J W and Kwok J C F (2013) Semaphorin 3A binds to the perineuronal nets via chondroitin sulfate type E motifs in rodent brains. J Biol Chem 288(38), 27384-395. (72 citations)
22\hat{ }Vo T*, Carulli D*, Ehlert E M E*, Kwok J C F*, Dick G, Mecollari V, Moloney E B, Neufeld G, de Winter F, Fawcett J W, Verhaagen J (2013) The chemorepulsive axon guidance protein semaphorin 3A is a constituent of perineuronal nets in the adult rodent brain. Mol Cell Neurosci 56C, 186-200. (67 citations)
21 Vorobyov V, Kwok J C, Fawcett J W, Sengpiel F. (2013) Effects of digesting chondroitin sulfate proteoglycans on plasticity in cat primary visual cortex. J Neurosci 33, 234-43. (40 citations)

2012
20\hat{ }Kwok J C, Yuen L Y, Lau, W K, Zhang F X, Fawcett J W, Chan Y S, Shum D K Y (2012) Chondroitin sulfates in the developing rat hindbrain confine commissural projections of vestibular nuclear neurons. Neural Dev. 7, 6-13.
19\hat{ }Kwok J C, Warren P, Fawcett J W (2012) Chondroitin sulfate: a key molecule in the brain matrix. Int J Biochem Cell Biol. 44,582-6. (83 citations)
18 Tan C L, Andrews M R, Kwok J C, Heintz T G P, Gumy L F, Fassler R, Fawcett J W (2012) Kindlin-1 enhances axon growth on inhibitory chondroitin sulfate proteoglycans and promotes sensory axon regeneration. J Neurosci 32, 7325-35. (43 citations)

2011
17\hat{ }Kwok J C, Tan C L, Wang D, Heller J, Fawcett J W (2011) Chondroitin sulfates in axon regeneration and plasticity. Trends in Glycosci and Glycotech 133, 201-11.
16\hat{ }Kwok J C, Dick D, Wang D, Fawcett J W (2011) Extracellular matrix and perineuronal nets in CNS repair. Dev Neurobiol 71, 1073-89. (250 citations)
15^{§} Lin R, Rosahl T W, Whiting P J, Fawcett J W, Kwok J C (2011) 6-Sulphated chondroitins have a positive influence on axonal regeneration. PLos One 6(7): e21499. (75 citations)
14 Zhao R R, Muir E M, Alves J N, Rickman H, Allan A Y, Kwok J C, Roet K C D, Verhaagen J, Schneider B L, Bensadoun J C, Ahmed S G, Yanez-Munoz R J, Keynes R J, Fawcett J W, Rogers J H (2011) Lentiviral vectors express Chondroitinase ABC in cortical projections and promote sprouting of injured corticospinal axons. J Neurosci Methods 201, 228-38. (55 citations)
13 Tan C L, Kwok J C, Patani R, Ffrench-Constant C, Chandran S, Fawcett J W (2011) Integrin activation promotes axon growth on inhibitory chondroitin sulfate proteoglycans by enhancing integrin signaling. J Neurosci 31, 6289-95. (112 citations)
12 Afshari F T, Kwok J C, Fawcett J W (2011) Analysis of Schwann-astrocyte interactions using in vitro assays. J Vis Exp. 47, 2214.doi: 10.3791/2214.

2010
11\hat{ }Kwok J C F, Carulli D, Fawcett J W (2010) In vitro modeling of perineuronal nets: the role of cartilage link protein and hyaluronan synthase in its formation and integrity. J Neurochem 114, 1447-59. (98 citations)
10 Hyatt A J T, Wang D, Kwok J C, Fawcett J W, Martin K R (2010) Controlled release of chondroitinase ABC from fibrin gel reduces the level of inhibitory glycosaminoglycan chains in lesioned spinal cord. J Control Release 147, 24-9. (66 citations)
9 Carulli D, Pizzorusso T, Kwok J C, Putignano E, Poli P, Forostyak S, Andrews M R, Deepa S S, Glant T T, Fawcett J W (2010) Animals lacking link protein have attenuated perineuronal nets and persistent plasticity. Brain 133, 2331-47. (278 citations)
8 Afshari F T, Kwok J C, Andrews M R, Blits B, Faissner A, Ffrench-Constant C, Fawcett J W (2010) Integrin activation or alpha-9 expression allows RPE cells to attach/migrate on Bruch's membrane in AMD. Brain 133, 448-64.
7 Afshari F T, Kwok J C, White L, Fawcett J W (2010) Schwann cell migration is integrin dependent and inhibited by astrocytes produced aggrecan. Glia 58, 857-69. (90 citations)
6 Afshari F T, Kwok J C, Fawcett J W (2010) Astrocyte-produced ephrins inhibit Schwann cell migration via VAV-2 signalling. J Neurosci 30, 4246-55. (63 citations)

2008
5\hat{ }Kwok J C, Afshari T, Garcia-Alias G, Fawcett J W (2008) Proteoglycans in the central nervous system: plasticity, regeneration and their stimulation with chondroitinase ABC. Restor Neurol Neurosci. 26, 131-45. (163 citations)
4\hat{ }Galtrey G M*, Kwok J C*, Carulli D, Rhodes K E, Fawcett J W (2008) Distribution and synthesis of extracellular matrix proteoglycans, hyaluronan, link proteins and tenascin-R in the rat spinal cord. Eur J Neurosci. 27, 1373-90. (150 citations)
3 Lin R, Kwok J C F, Crespo D, Fawcett J W (2008) Chondroitinase ABC has a long-lasting effect on chondroitin sulphate glycosaminoglycans content in the injured rat brain. J Neurochem. 104, 400-8. (81 citations)
2 Gervasi N M, Kwok J C, Fawcett J W (2008) Role of extracellular factors in axon regeneration in the CNS: implications for therapy. Regen Med 3, 907-23. (51 citations)
1 Properzi F, Lin R, Kwok J C, Naidu M, van Kuppevelt T H, ten Dam G B, Camargo L M, Raha-Chowdhury R, Furukawa Y, Mikami T, Sugahara K, Fawcett J W (2008) Heparan sulphate proteoglycans in glia and in the normal and injured CNS: Expression of sulphotransferases and changes in sulphation. Eur J

2019
47^{§} Duncan J A, Foster R, Kwok J C F (2019) The potential of memory enhancement through perineuronal net modulation. Brit J Pharmacol (accepted)
45 Tsatsanis A, Dickens S, Kwok J C F, Wong B, Duce J A (2019) Post translational modulation of β-amyloid precursor protein trafficking to the cell surface alters neuronal iron homeostasis. Neurochem Res (accepted)

2018
44^{§} Warren P M, Dickens S M, Gigout S, Fawcett J W and Kwok J C F (2018) Regulation of CNS plasticity through the extracellular matrix. Oxford Handbook of Developmental Neural Plasticity. (accepted)
43^{§} Irvine S F and Kwok J C F (2018) Perineuronal nets in spinal motoneurones: chondroitin sulphate proteoglycan around alpha motoneurones. Int J Mol Sci 19, 1172-90.
42^{§} Richter R P*, Baranovab N S, Day A J, Kwok J C F* (2018) Glycosaminoglycans in extracellular matrix organisation: Are concepts from soft matter physics key to understanding the formation of perineuronal nets? Curr Opin Struct Biol 50, 65-74.

2017
41^{§} van't Spijker H M and Kwok J C F (2017) A sweet talk: the molecular systems of perineuronal nets in controlling neuronal communication. Front Integr Neurosci 11, 33.
40 Koseki H, Donega M, Lam B Y H, Petrova V, van Erp S, Yeo G S H, Kwok J C F, ffrench-Constant C, Eva R, Fawcett J W (2017) Selective Rab11 transport and the intrinsic regenerative ability of CNS axons. eLife 6, e26956.
39^{§} Yang S, Alves J N, Hilton S, Saksida L M, Bussey T M, Matthews R T, Kitagawa H, Spillantini M G, Kwok J C F*, Fawcett J W* (2017) Antibody recognizing 4-sulphated chondroitin sulphate proteoglycans restores memory in tauopathy-induced neurodegeneration. Neurobiol Aging 59, 197-209.
38 Kempf A, Fritz R, Boda E, Kwok J C F, Kaelin A M, Schmandke A, Ristic Z, Tews B, Fawcett J W, Buffo A, Pertz O, Schwab M E. (2017) Control of cell shape, neurite outgrowth and migration by a novel Nogo-A/HSPG interaction. Dev Cell S1534-5807, 30674-3.
37^{§ \hat{ }} Foscarin S, Fawcett J W, Kwok J C F (2017) Brain ageing changes proteoglycan sulphation, rendering perineuronal nets more inhibitory. Aging-US 9(6), 1607-1622.
36^{§ \hat{ }} Djerbal L, Lortat-Jacob H*, Kwok J C F* (2017) Chondroitin sulfates and their binding molecules in the central nervous system. Glycoconj J 34(3):363-376.

2016
35 Sorg B A, Berretta S, Blacktop J M, Fawcett J W, Kitagawa H, Kwok J C F, Miquel M (2016) Casting a wide net: role of perineuronal nets in neural plasticity. J Neurosci 36, 11459-11468. (51 citations)
34^{§} Vecino E, Kwok J C F (2016) The Extracellular Matrix in the Nervous System: The Good and the Bad Aspects. InTech 2016, 95-114.
33 Carulli D, Kwok J C F, Pizzorusso T (2016) Perineuronal nets and CNS plasticity and repair. Neural Plasticity 2016, 4327082.
32 de Winter F, Kwok J C F, Fawcett J W, Vo T T, Carulli D and Verhaagen J (2016) The chemorepulsive protein semaphorin 3A and perineuronal net-mediated plasticity. Neural Plasticity 2016, 3679545.

2015
31 Heller J P, Kwok J C F, Vecino E, Martin K R and Fawcett J W (2015) A method for the isolation and culture of adult rat retinal pigment epithelial (RPE) cells to study retinal diseases. Front Cell Neurosci 9, 449.
30 Smith P D, Coulson-Thomas V J, Foscarin S, Kwok J C F and Fawcett J W (2015) “GAG-ing with the neuron”: The role of glycosaminoglycan patterning in the central nervous system. Exp Neurol. 274(Pt B), 100-14.
29 Tan C L, Kwok J C F, Heller J P D, Zhao R, Eva R, Fawcett J W (2015) Full length talin stimulates integrin activation and axon regeneration. Mole Cell Neuro 68, 1-8.
28^{§} Rowlands D, Sugahara K, Kwok J C F (2015) Glycosaminoglycans and glycomimetics in the central nervous system. Molecules 20, 3527-48.

2014
27\hat{ } Yang S*, Kwok J C*, Fawcett J W (2014) Neural ECM in Regeneration and Rehabilitation. Prog in Brain Res 214, 179-92.
26 Tyzack G E, Sitnikov S, Barson D, Adams-Carr K L, Lau N K, Kwok J C, Zhao C, Franklin R J, Karadottir R T, Fawcett J W, Lakatos A (2014) Astrocyte response to motor neuron injury promotes structural synaptic plasticity via STAT3-regulated TSP-1 expression. Nat Commun 5, 4294. (49 citations)
25\hat{ } Kwok J C F, Heller J P, Zhao R R, Fawcett J W (2014) Targeting inhibitory chondroitin sulphate proteoglycans to promote plasticity after injury. Axon Growth and Regeneration: Methods Mol Biol
Andreichenko, I. N., A. A. Tsitrina, A. V. Fokin, A. I. Gabdulkhakova, D. I. Maltsev, G. S. Perelman, E. V. Bulgakova, A. M. Kulikov, A. S. Mikaelyan, and Y. V. Kotelevtsev. 2019. 4-methylumbelliferone Prevents Liver Fibrosis by Affecting Hyaluronan Deposition, FSTL1 Expression and Cell Localization. International journal of molecular sciences. 20:6301.
Basso, D. M., M. S. Beattie, and J. C. Bresnahan. 1995. A sensitive and reliable locomotor rating scale for open field testing in rats. Journal of neurotrauma. 12:1-21.
Bradbury, E. J., L. D. F. Moon, R. J. Popat, V. R. King, G. S. Bennett, P. N. Patel, J. W. Fawcett, and S. B. McMahon. 2002. Chondroitinase ABC promotes functional recovery after spinal cord injury. Nature. 416:636-640.
Carulli, D., T. Pizzorusso, J. C. Kwok, E. Putignano, A. Poli, S. Forostyak, M. R. Andrews, S. S. Deepa, T. T. Glant, and J. W. Fawcett. 2010. Animals lacking link protein have attenuated perineuronal nets and persistent plasticity. Brain. 133:2331-2347.
Chaplan, S. R., F. W. Bach, J. W. Pogrel, J. M. Chung, and T. L. Yaksh. 1994. Quantitative assessment of tactile allodynia in the rat paw. Journal of neuroscience methods. 53:55-63.
de Winter, F., J. C. Kwok, J. W. Fawcett, T. T. Vo, D. Carulli, and J. Verhaagen. 2016. The Chemorepulsive Protein Semaphorin 3A and Perineuronal Net-Mediated Plasticity. Neural plasticity. 2016:3679545.
Dixon, W. J. 1980. Efficient analysis of experimental observations. Annual review of pharmacology and toxicology. 20:441-462.
Fontaine, L., Grand, M., Chabert, J., Molho, D., Boschetti, E. 1968. Etude toxicologigue et teratologique de la methy 4 ombelliferone (1). Therapie. XXIII:359-371.
Gage, G. J., D. R. Kipke, and W. Shain. 2012. Whole animal perfusion fixation for rodents. J Vis Exp.
Galtrey, C. M., J. C. Kwok, D. Carulli, K. E. Rhodes, and J. W. Fawcett. 2008. Distribution and synthesis of extracellular matrix proteoglycans, hyaluronan, link proteins and tenascin-R in the rat spinal cord. Eur J Neurosci. 27:1373-1390.
Garcia-Alias, G., S. Barkhuysen, M. Buckle, and J. W. Fawcett. 2009. Chondroitinase ABC treatment opens a window of opportunity for task-specific rehabilitation. Nat Neurosci. 12:1145-1151.
Garcia-Alias, G., H. A. Petrosyan, L. Schnell, P. J. Horner, W. J. Bowers, L. M. Mendell, J. W. Fawcett, and V. L. Arvanian. 2011. Chondroitinase ABC combined with neurotrophin NT-3 secretion and NR2D expression promotes axonal plasticity and functional recovery in rats with lateral hemisection of the spinal cord. J Neurosci. 31:17788-17799.
Gigout, S., J. Louvel, D. Rinaldi, B. Martin, and R. Pumain. 2013. Thalamocortical relationships and network synchronization in a new genetic model “in mirror” for absence epilepsy. Brain Research. 1525:39-52.
Hooven, M. D. 2017. Opportunities and Challenges in Biologic Drug Delivery. American Pharmaceutical Review.
Irvine, S., and J. Kwok. 2018. Perineuronal Nets in Spinal Motoneurones: Chondroitin Sulphate Proteoglycan around Alpha Motoneurones. International Journal of Molecular Sciences. 19:1172.
Kwok, J. C., G. Dick, D. Wang, and J. W. Fawcett. 2011. Extracellular matrix and perineuronal nets in CNS repair. Dev Neurobiol. 71:1073-1089.
Kwok, J. C. F., D. Carulli, and J. W. Fawcett. 2010. In vitro modeling of perineuronal nets: hyaluronan synthase and link protein are necessary for their formation and integrity. Journal of neurochemistry. 114:1447-1459.
Luhmann, H. J., T. Mittmann, G. van Luijtelaar, and U. Heinemann. 1995. Impairment of intracortical GABAergic inhibition in a rat model of absence epilepsy. Epilepsy Research. 22:43-51.
Metz, G. A., and I. Q. Whishaw. 2009. The ladder rung walking task: a scoring system and its practical application. J Vis Exp:1204.
Nagy, N., H. F. Kuipers, A. R. Frymoyer, H. D. Ishak, J. B. Bollyky, T. N. Wight, and P. L. Bollyky. 2015. 4-Methylumbelliferone Treatment and Hyaluronan Inhibition as a Therapeutic Strategy in Inflammation, Autoimmunity, and Cancer. Frontiers in Immunology. 6.
Nakamura, T., K. Takagaki, S. Shibata, K. Tanaka, T. Higuchi, and M. Endo. 1995. Hyaluronic-Acid-Deficient Extracellular Matrix Induced by Addition of 4-Methylumbelliferone to the Medium of Cultured Human Skin Fibroblasts. Biochemical and Biophysical Research Communications. 208:470-475.
Pizzorusso, T., P. Medini, N. Berardi, S. Chierzi, J. W. Fawcett, and L. Maffei. 2002. Reactivation of ocular dominance plasticity in the adult visual cortex. Science. 298:1248-1251.
Silver, J., and J. H. Miller. 2004. Regeneration beyond the glial scar. Nat Rev Neurosci. 5:146-156.
Vo, T., D. Carulli, E. M. E. Ehlert, J. C. F. Kwok, G. Dick, V. Mecollari, E. B. Moloney, G. Neufeld, F. de Winter, J. W. Fawcett, and J. Verhaagen. 2013. The chemorepulsive axon guidance protein semaphorin3A is a constituent of perineuronal nets in the adult rodent brain. Molecular and Cellular Neuroscience. 56:186-200.
Wang, D., R. M. Ichiyama, R. Zhao, M. R. Andrews, and J. W. Fawcett. 2011. Chondroitinase combined with rehabilitation promotes recovery of forelimb function in rats with chronic spinal cord injury. J Neurosci. 31:9332-9344.
Warren, P. M., S. C. Steiger, T. E. Dick, P. M. MacFarlane, W. J. Alilain, and J. Silver. 2018. Rapid and robust restoration of breathing long after spinal cord injury. Nature Communications. 9:4843.

USPTO References
U.S. Pat. Nos. 9,415,218; 9,101,769; 9,393,409; 9,409,023; 7,326,649; 8,805,542; 9,409,011; 62/800,817; 62/828,853
M G Fehlings et al Traumatic spinal cord injury Nat. Rev. Dis. Primers 3. 17018 (2017) Ros Lynch Neurological Report 21 Aug. 2018 JSuggitt MS OTR/L CLT

1. 4-methylumbelliferone, a derivative or salt thereof, for use in the treatment of a condition of the nervous system in a subject.

2. A pharmaceutical composition comprising a therapeutically effective amount of 4-methylumbelliferone, a derivative or salt thereof, for use in the treatment of a condition of the nervous system.

3. The 4-methylumbelliferone, derivative or salt thereof, for use according to claim 1, or the pharmaceutical composition for use according to claim 2, wherein the condition of the nervous system is one associated with a lesion.

4. The 4-methylumbelliferone, derivative or salt thereof, for use according to claim 3, or the pharmaceutical composition for use according to claim 3, wherein the lesion is selected from the group comprising a glial scar, an amyloid lesion, tau aggregates and Lewy bodies.

5. The 4-methylumbelliferone, derivative or salt thereof, for use according to claim 4, or the pharmaceutical composition for use according to claim 4, wherein the lesion is a glial scar.

6. The 4-methylumbelliferone, derivative or salt thereof, for use according to claims 1, 3 to 5, or the pharmaceutical composition for use according to claims 2 to 5, wherein the condition of the nervous system is selected from the group comprising conditions caused by trauma, injury, infection, degeneration, structural defects, tumours, blood flow disruption.

7. The 4-methylumbelliferone, derivative or salt thereof, for use according to claim 6, or the pharmaceutical composition for use according to claim 6, wherein the condition may be selected from the group comprising stroke, transient ischemic attach, haemorrhage, meningitis, encephalitis, bell's palsy, brain or spinal tumour, Parkinson's disease, Huntington chorea, and Alzheimer disease.

8. The 4-methylumbelliferone, derivative or salt thereof, for use according to claim 7 or the pharmaceutical composition for use according to claim 7, wherein the condition is a spinal cord injury.

9. The 4-methylumbelliferone, derivative or salt thereof, for use according to claim 8 or the pharmaceutical composition for use according to claim 8, wherein the 4-methylumbelliferone, derivative or salt thereof, or the pharmaceutical composition, is to be administered immediately after injury, or within the first seven days after injury and optionally, is to be followed by continuous treatment.

10. The 4-methylumbelliferone, derivative or salt thereof, for use according to claim 8 or 9, or the pharmaceutical composition for use according to claim 8 or 9, wherein the 4-methylumbelliferone, derivative or salt thereof, or the pharmaceutical composition is to be administered in combination with rehabilitation or electrostimulation or a combination thereof.

11. The 4-methylumbelliferone, derivative or salt thereof, for use according to any one of claim 1, or 3 to 10, or the pharmaceutical composition for use according to any one of claims 2 to 10, wherein the 4-methylumbelliferone, derivative or salt thereof, or the pharmaceutical composition, is formulated for oral delivery.

12. The 4-methylumbelliferone, derivative or salt thereof, for use according to any one of claim 1, or 3 to 10, or the pharmaceutical composition for use according to any one of claims 2 to 10, wherein the 4-methylumbelliferone, derivative or salt thereof, or the pharmaceutical composition, is formulated for injection, preferably injection directly into the scar or spinal cord of said subject.

13. The 4-methylumbelliferone, derivative or salt thereof, for use according to any one of claim 1, or 3 to 12, or the pharmaceutical composition for use according to any one of claims 2 to 12, wherein the 4-methylumbelliferone derivative is a molecule of the formula: 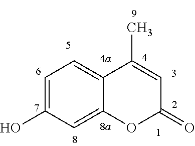
wherein, one or more alkyl, aryl, acyl, dimethyamino, hydroxyl, hydroxymethyl, methoxy, methyl, morpholino and sulfonyl substituents is added at one or more positions C1 to C9.

14. The 4-methylumbelliferone, derivative or salt thereof, for use according to claim 13, or the pharmaceutical composition for use according to claim 13, wherein NR₁R₂ is added to the methyl group (CH₃) at C4, and wherein R₁ and/or R₂ are each independently be H, alkyl, aryl, acyl or sulfonyl.

15. The 4-methylumbelliferone, derivative or salt thereof, for use according to claim 14, or the pharmaceutical composition for use according to claim 14, wherein R₁ and R₂ are alkyl.

16. The 4-methylumbelliferone, derivative or salt thereof, for use according to claim 13, or the pharmaceutical composition for use according to claim 13, wherein OR₁ is added to the methyl group (CH₃) at C4, and wherein R₁ is alkyl, aryl or acyl.

17. The 4-methylumbelliferone, derivative or salt thereof, for use according to claim 16, or the pharmaceutical composition for use according to claim 16, wherein R₁ is hydroxyethyl.

18. The 4-methylumbelliferone, derivative or salt thereof, for use according to claim 13, or the pharmaceutical composition for use according to claim 13, wherein C3, C5, C6 and or C8, are substituted with one or more alkyl, aryl, acyl, dimethyamino, hydroxyl, hydroxymethyl, methoxy, methyl, morpholino and sulfonyl substituents.

19. The 4-methylumbelliferone, derivative or salt thereof, for use according to claim 13, or the pharmaceutical composition for use according to claim 13, wherein O at position 1 is replaced with NR₁ and wherein R₁ is aryl or acyl.

20. The 4-methylumbelliferone, derivative or salt thereof, for use according to claim 19, or the pharmaceutical composition for use according to claim 19, wherein R₁ is alkyl.

21. The 4-methylumbelliferone, derivative or salt thereof, for use according to any one of claim 1, or 3 to 20, or the pharmaceutical composition for use according to any one of claims 2 to 20, wherein the 4-methylumbelliferone, derivative or salt thereof, or the pharmaceutical composition, is to be administered at a dose of from 5 to 60 mg/kg body weight per day.

22. The 4-methylumbelliferone, derivative or salt thereof, for use according to claim 21, or the pharmaceutical composition for use according to claim 21, wherein the dose is from about 500 to 600 mg at least two times per day.

23. A 4-methylumbelliferone, derivative or salt thereof, for use in a method of treating a lesion associated with a condition of the nervous system.

24. The 4-methylumbelliferone, derivative or salt thereof, for use according to claim 23 wherein the lesion is a glial scar.

25. The 4-methylumbelliferone, derivative or salt thereof, for use according to claim 23, or 24, wherein the condition of the nervous system is selected from the group comprising conditions caused by trauma, injury, infection, degeneration, structural defects, tumours, blood flow disruption.

26. The 4-methylumbelliferone, derivative or salt thereof, for use according to claim 25, in wherein condition may be selected from the group comprising stroke, transient ischemic attach, haemorrhage, meningitis, encephalitis, bell's palsy, brain or spinal tumour, Parkinson's disease, Huntington chorea, Alzheimer disease and cerebral palsy.

27. The 4-methylumbelliferone, derivative or salt thereof, for use according to claim 26, wherein the condition is a spinal cord injury.

28. The 4-methylumbelliferone, derivative or salt thereof, for use according to any one of claims 23 to 28, wherein the 4-methylumbelliferone derivative is a molecule of the formula: 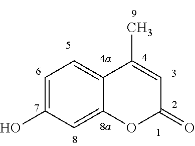
wherein, one or more alkyl, aryl, acyl, dimethyamino, hydroxyl, hydroxymethyl, methoxy, methyl, morpholino and sulfonyl substituents is added at one or more positions C1 to C9.

29. A 4-methylumbelliferone derivative or salt thereof, of the formula: 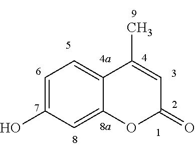
wherein, one or more alkyl, aryl, acyl, dimethyamino, hydroxyl, hydroxymethyl, methoxy, methyl, morpholino and sulfonyl substituents is added at one or more positions C1 to C9.

30. The 4-methylumbelliferone derivative according to claim 29, wherein NR₁R₂ is added to the methyl group (CH₃) at C4, and wherein R₁ and/or R₂ are each independently be H, alkyl, aryl, acyl or sulfonyl.

31. The 4-methylumbelliferone derivative according to claim 30, wherein R₁ and R₂ are alkyl.

32. The 4-methylumbelliferone derivative according to claim 29, wherein OR₁ is added to the methyl group (CH₃) at C4, and wherein R₁ is alkyl, aryl or acyl.

33. The 4-methylumbelliferone derivative according to claim 32, wherein R¹ is hydroxyethyl.

34. The 4-methylumbelliferone derivative according to claim 29, wherein O at position 1 is replaced with NR₁ and wherein R₁ is aryl or acyl.

35. The 4-methylumbelliferone derivative according to claim 34, wherein R₁ is alkyl.
